(19) United States
(12) Patent Application Publication
(10) Pub. No.: US 2007/0003956 A1
(43) Pub. Date: Jan. 4, 2007

(54) APPROACHES TO IDENTIFYING GENETIC TRAITS IN ANIMALS

(75) Inventors: Max F. Rothschild, Ames, IA (US); Stefan Marklund, Uppsala (SE); Richard M. Robson, Ames, IA (US); Ted W. Huiatt, Ames, IA (US); Jeannine M. Helm, Bethesda, MD (US); Tun-Ping Yu, Franklin, KY (US); Graham S. Plastow, Franklin, KY (US)

Correspondence Address: MCKEE, VOORHEES & SEASE, P.L.C., 801 GRAND AVENUE, SUITE 3200, DES MOINES, IA, 50309-2721, US

(73) Assignees: IOWA STATE UNIVERSITY RESEARCH FOUNDATION, INC., Ames, IA (US); PIG IMPROVEMENT COMPANY UK LIMITED, Abingdon (GB)

(21) Appl. No.: 11/466,968

(22) Filed: Aug. 24, 2006

Related U.S. Application Data
(63) Continuation of application No. 10/798,678, filed on Mar. 11, 2004.
(60) Provisional application No. 60/453,752, filed on Mar. 11, 2003.

(51) Int. Cl. C12Q 1/68; C12P 19/34

(52) U.S. Cl. 435/6; 435/91.2

(57) ABSTRACT

Disclosed herein are embodiments to screen an animal for the presence or absence of polymorphisms in the following gene: CKM, SCN4α, and LDHα.

### CROSS-REFERENCE TO RELATED APPLICATIONS

This application is a continuation of U.S. patent application Ser. No. 10/798,678 filed Mar. 11, 2004, which is a nonprovisional application of U.S. Provisional Application No. 60/453,752, filed Mar. 11, 2003, which are herein incorporated by reference in their entirety.

### GRANT REFERENCE

This invention was supported at least in part by USDA/CREES Contract Nos. 99-CRHF-0-6019, and 98-CRHR-0-6019 (IAHAEES project number 3148). The United States government may have certain rights in this invention.

### BACKGROUND OF THE INVENTION

Genetic mutations are the basis of evolution and genetic diversity. Genetic markers represent specific loci in the genome of a species, population or closely related species, and sampling of different genotypes at these marker loci reveals genetic variation. The genetic variation at marker loci can then be described and applied to genetic studies, commercial breeding, diagnostics, and cladistic. Genetic markers have the greatest utility when they are codominant, highly heritable, multi-allelic, and numerous. Most genetic markers are heritable because their alleles are determined by the nucleotide sequence of DNA which is highly conserved from one generation to the next, and the detection of their alleles is unaffected by the natural environment. Markers have multiple alleles because, in the evolutionary process, rare, genetically-stable mutations in DNA sequences defining marker loci arose and were disseminated through the generations along with other existing alleles. The highly conserved nature of DNA combined with rare occurrences of stable mutations allows genetic markers to be both predictable and discerning of different genotypes. The repertoire of genetic-marker technologies today allows multiple technologies to be used simultaneously in the same project. The invention of each new genetic-marker technology and each new DNA polymorphism adds additional utility to genetic markers. Many genetic-marker technologies exist. Some examples are restriction-fragment-length polymorphism (RFLP) Bostein et al (1980) Am J Hum Genet 32:314-331; single-strand conformation polymorphism (SSCP) Fischer et al. (1983) Proc Natl Acad Sci USA 80:1579-1583, Orita et al. (1989) Genomics 5:874-879; amplified fragment-length polymorphism (AFLP) Vos et al. (1995) Nucleic Acids Res 23:4407-4414; microsatellite or single-sequence repeat (SSR) Weber J L and May P E (1989) Am J Hum Genet 44:388-396; rapid-amplified polymorphic DNA (RAPD) Williams et al (1990) Nucleic Acids Res 18:6531-6535; sequence tagged site (STS) Olson et al. (1989) Science 245:1434-1435; genetic-bit analysis (GBA) Nikiforov et al (1994) Nucleic Acids Res 22:4167-4175; allele-specific polymerase chain reaction (ASPCR) Gibbs et al. (1989) Nucleic Acids Res 17:2437-2448, Newton et al. (1989) Nucleic Acids Res 17:2503-2516; nick-translation PCR (e.g., TAQMAN™) Lee et al. (1993) Nucleic Acids Res 21:3761-3766; and allele-specific hybridization (ASH) Wallace et al. (1979) Nucleic Acids Res 6:3543-3557, (Sheldon et al. (1993) Clinical Chemistry 39(4):718-719) among others. Each technology has its own particular basis for detecting polymorphisms in DNA sequence.

Genetic differences exist among individual animals as well as among breeds which can be exploited by breeding techniques to achieve animals with desirable characteristics. For example, Chinese breeds are known for reaching puberty at an early age and for their large litter size, while American breeds are known for their greater growth rates and leanness. However, heritability for desired traits is often low, and standard breeding methods which select individuals based upon phenotypic variations do not take fully into account genetic variability or complex gene interactions which exist.

Restriction fragment length polymorphism (RFLP) analysis has been used by several groups to study pig DNA. Jung et al., Theor. Appl. Genet., 77:271-274 (1989), incorporated herein by reference, discloses the use of RFLP techniques to show genetic variability between two pig breeds. Polymorphism was demonstrated for swine leukocyte antigen (SLA) Class I genes in these breeds. Hoganson et al., Abstract for Annual Meeting of Midwestern Section of the American Society of Animal Science, Mar. 26-28, 1990, incorporated herein by reference, reports on the polymorphism of swine major histocompatibility complex (MHC) genes for Chinese pigs, also demonstrated by RFLP analysis. Jung et al., Theor. Appl. Genet., 77:271-274 (1989), incorporated herein by reference, reports on RFLP analysis of SLA Class I genes in certain boars. The authors state that the results suggest that there may be an association between swine SLA/MHC Class I genes and production and performance traits. They further state that the use of SLA Class I restriction fragments, as genetic markers, may have potential in the future for improving pig growth performance.

The ability to follow a specific favorable genetic allele involves a novel and lengthy process of the identification of a DNA molecular marker for a major effect gene. The marker may be linked to a single gene with a major effect or linked to a number of genes with additive effects. DNA markers have several advantages; segregation is easy to measure and is unambiguous, and DNA markers are co-dominant, i.e., heterozygous and homozygous animals can be distinctively identified. Once a marker system is established selection decisions could be made very easily, since DNA markers can be assayed any time after a tissue or blood sample can be collected from the individual infant animal, or even an embryo.

The use of genetic differences in receptor genes has become a valuable marker system for selection. For example, U.S. Pat. Nos. 5,550,024 and 5,374,526 issued to Rothschild et al. disclose a polymorphism in the pig estrogen receptor gene which is associated with larger litter size, the disclosure of which is incorporated herein by reference. U.S. Pat. No. 5,935,784 discloses polymorphic markers in the pig prolactin receptor gene which are associated with larger litter size and overall reproductive efficiency.

The quality of raw pig meat is influenced by a large number of genetic and non-genetic factors. The latter include farm, transport, slaughter and processing conditions. Meat scientists have performed a substantial amount of research on these factors, which has led to considerable quality improvement. Part of the research has also been dedicated to the genetic background of the animals, and several studies have revealed the importance of genetic factors. This has made the industry aware that selective breeding of animals and the use of gene technology can play an important role in enhancing pork quality.

Information at the DNA level can help to fix a specific major gene, but it can also assist the selection of a quantitative trait for which we already select. Molecular information in addition to phenotypic data can increase the accuracy of selection and therefore the selection response. The size of the extra response in such a Marker Assisted Selection (MAS) program has been considered by many workers from a theoretical point of view. In general terms, MAS is more beneficial for traits with a low heritability and which are expensive to measure phenotypically. Although traits such as meat quality and/or growth are not typically considered in this way there are still significant advantages for the use of markers for these traits. For example, Meuwissen and Goddard considered the impact of MAS for different types of traits. The biggest impacts were for traits such as meat quality, where the trait is measured after slaughter and an additional response of up to 64% could be achieved with the incorporation of marker information for selection on the animals before slaughter. This figure was relatively small, 8%, for growth traits, that can be measured on the live animal. However, once the association has been demonstrated this marker information can be used before the animals are tested or selected phenotypically (see below) and in this situation a response of up to 38% was predicted.

Indeed, the best approach to genetically improve economic traits is to find relevant DNA-markers directly in the population under selection. Phenotypic measurements can be performed continuously on some animals from the nucleus populations of breeding organizations. Since a full assessment of most of these traits can only be done after slaughter, the data must be collected on culled animals and cannot be obtained on potential breeding animals.

This phenotypic data is collected in order to enable the detection of relevant DNA markers, and to validate markers identified using experimental populations or to test candidate genes. Significant markers or genes can then be included directly in the selection process. An advantage of the molecular information is that we can obtain it already at very young age of the breeding animal, which means that animals can be preselected based on DNA markers before the growing performance test is completed. This is a great advantage for the overall testing and selection system.

Accordingly, there exists a need for candidate genes and genetic markers for genotyping, for identity conservation, for marker assisted selection, genetic studies and positional cloning of nucleic acids in animals.

Therefore, it is an object of the present invention to provide candidate genes and genetic markers based on or within these genes which are which are indicative of favorable economic characteristics. These candidate genes are selected from the following: creatine kinase-muscle (CKM), the sodium channel, voltage gated, type IV alpha gene (SCN4α), and the lactate dehydrogenase alpha gene (LDHα) gene.

Another object of the invention is to provide an assay for determining the presence of these genetic markers.

A further object of the invention is to provide a method of evaluating animals that increases accuracy of selection and breeding methods for the desired traits.

Yet another object of the invention is to provide a PCR amplification test which will greatly expedite the determination of presence of the markers.

Additional objects and advantages of the invention will be set forth in part in the description that follows, and in part will be obvious from the description, or may be learned by the practice of the invention. The objects and advantages of the invention will be attained by means of the instrumentalities and combinations particularly pointed out in the appended claims.

### BRIEF SUMMARY OF THE INVENTION

The present invention relates to the discovery of alternate gene forms in various porcine genes which are useful in identifying favorable genetic traits for animal breeding. To the extent that this gene is conserved among species and animals, and it is expected that the different alleles disclosed herein will also correlate with variability in this gene in other economic or meat-producing animals such as bovine, sheep, chicken, etc. Identification of these alleles provides methods for rapidly determining the genotype of an animal. This ability to determine, accurately and quickly, the genotype of an animal provides for improved methods of marker assisted selection in animal breeding and analysis of a plurality of animals.

According to the present invention there are provided methods for identifying the presence or absence of a polymorphism in an animal. Another embodiment includes a method of determining an animal's genetic potential for animal breeding. A further embodiment includes a method of screening animals to determine those more likely to possess favorable meat quality traits, those with heavy muscling and/or those who may have skeletal muscle cramping disease. These methods include obtaining a genetic sample from the animal. The methods can further include assaying for the presence or absence of a polymorphism in a gene associated with improved meat quality, heavy muscling and/or skeletal muscle cramping disease wherein the gene is selected from a group consisting of creatine kinase-muscle (CKM), the sodium channel, voltage gated, type IV alpha gene (SCN4α), and the lactate dehydrogenase alpha gene (LDHα or LDHA). Such assays can be restriction fragment length polymorphisms (RFLP), heteroduplex analysis, single-strand conformational polymorphism, denaturing gradient gel electrophoresis (DGGE) and temperature gel electrophoresis (TGGE).

The methods of the present invention can further include making, for example, meat quality and heavy muscling assessments based upon the presence or absence of a genotype in the animal wherein the genotype is correlated with these traits.

Another embodiment includes genotyping an animal to determining those with a favorable combination of alleles associated with traits such as favorable meat quality, heavy muscling and/or increased likelihood of skeletal muscle cramping disease or alternatively against those animals carrying unfavorable combinations of alleles.

Further, the embodiments of the present invention can include amplifying an amount of the gene or a portion thereof, which contains the polymorphism.

Factors for meat quality which may be considered include but are not limited to the following:

Loin Minolta Lightness (L*): The range of 43-47 units (from darker to lighter color) is acceptable, but L* of 43 is better; i.e., has higher economic value, in general in this range (this may be dependent upon market, for example in Japan darker pork is preferred).

Loin Japanese Color Score (JCS): The range of 2.5-5.0 units (from lighter to darker color) is acceptable, but JCS of 3-4 is better.

Loin Marbling (level of intramuscular fat): Generally, higher marbling is better as it is associated with improved meat eating quality characteristics.

Loin pH: (ultimate meat acidity measured 24 hours post-mortem; this attribute is the single most important trait of pork quality); The range of 5.50-5-80 is desirable, but 5.80 is better as it positively influences the color and (low) purge of the meat.

Ham Minolta lightness (L*): The range of 43-52 units is acceptable, but lower (43) is better.

Ham pHu: higher; i.e., 5.80, is better.

Drip loss or purge: the range of 1%-3% is acceptable, but lower is better.

These measures of meat quality are examples of those generally accepted by those of skill in the art. For a review of meat quality traits the following may be consulted: Sosnicki, A. A., E. R. Wilson, E. B. Sheiss, A. deVries, 1998 “Is there a cost effective way to produce high quality pork?”, Reciprocal Meat Conference Proceedings, Vol. 51.

Growth can be measured by any of a number of standard means such as average daily gain, weight at slaughter, etc.

Since several of the polymorphisms may involve changes in amino acid composition of the CKM, SCN4α, and the LDHα protein, assay methods may even involve ascertaining the amino acid composition of these proteins. Methods for this type or purification and analysis typically involve isolation of the protein through means including fluorescence tagging with antibodies, separation and purification of the protein (i.e., through reverse phase HPLC system), and use of an automated protein sequencer to identify the amino acid sequence present. Protocols for this assay are standard and known in the art and are disclosed in Ausubel et. al. (eds.), Short Protocols in Molecular Biology 4th ed. (John Wiley and Sons 1999).

In a preferred embodiment a genetic sample is analyzed. Briefly, a sample of genetic material is obtained from an animal, and the sample is analyzed to determine the presence or absence of a polymorphism in the CKM, SCN4α, or LDHα gene, which are correlated with meat quality, heavy muscling, and/or skeletal muscle cramping disease depending on the gene form.

As is well known to those of skill in the art, a variety of techniques may be utilized when comparing nucleic acid molecules for sequence differences. These include by way of example, restriction fragment length polymorphism analysis, heteroduplex analysis, single-strand conformation polymorphism analysis, denaturing gradient electrophoresis and temperature gradient electrophoresis.

In a preferred embodiment the polymorphism is a restriction fragment length polymorphism and the assay comprises identifying the animal's CKM, SCN4α, or LDHα gene from isolated genetic material; exposing the gene to a restriction enzyme that yields restriction fragments of the gene of varying length; separating the restriction fragments to form a restriction pattern, such as by electrophoresis or HPLC separation; and comparing the resulting restriction fragment pattern from a CKM, SCN4α, or LDHα gene that is either known to have or not to have the desired marker. If an animal tests positive for the preferred markers, such animal can be considered for inclusion in the breeding program. If the animal does not test positive for the preferred marker genotype the animal can be culled from the group and otherwise used. Use of haplotype data can also be incorporated with the screening for multiple alleles for different aspects of meat quality, heavy muscling, and/or skeletal muscle cramping disease.

In a most preferred embodiment the gene is isolated by the use of primers and DNA polymerase to amplify a specific region of the gene which contains the polymorphism. Next the amplified region is digested with a restriction enzyme and fragments are again separated. Visualization of the RFLP pattern is by simple staining of the fragments, or by labeling the primers or the nucleoside triphosphates used in amplification. In another embodiment, the invention comprises screening animals to determine the animal's genetic potential. A polymorphism in the CKM, SCN4α, or LDHα gene of each pig is identified and associated with the meat quality, heavy muscling, and/or skeletal muscle cramping disease. Preferably, RFLP analysis is used to determine the polymorphism.

In another embodiment, the invention comprises a method for identifying a genetic marker for meat quality, heavy muscling, and/or skeletal muscle cramping disease in any particular economic animal other than a pig. Based upon the highly conserved nature of this gene among different animals and the location of the polymorphisms within these highly conserved regions, is it expected that with no more than routine testing as described herein that these markers can be applied to different animal species to select for meat quality, heavy muscling, and/or skeletal muscle cramping disease based on the teachings herein. Male and female animals of the same breed or breed cross or similar genetic lineage are bred, and the meat quality, heavy muscling, and/or skeletal muscle cramping disease produced by each animal is determined and correlated. For other animals in which sequences are available a BLAST comparison of sequences may be used to ascertain whether the particular allele is analogous to the one disclosed herein. The analogous polymorphism will be present in other animals and in other closely related genes. The term “analogous polymorphism” shall be a polymorphism which is the same as any of those disclosed herein as determined by BLAST comparisons.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. In this case the reference sequences are CKM, SCN4α, and LDHα. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information (http://www.hcbi.nlm.nih.gov/).

This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol Sci., 4:11-17 (1988) e.g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e) The term “substantial identity” of polynucleotide sequences means that a polynucleotide comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or preferably at least 70%, 80%, 90%, and most preferably at least 95%.

These programs and algorithms can ascertain the analogy of a particular polymorphism in a target gene to those disclosed herein. It is expected that this polymorphism will exist in other animals and use of the same in other animals than disclosed herein involved no more than routine optimization of parameters using the teachings herein.

It is also possible to establish linkage between specific alleles of alternative DNA markers and alleles of DNA markers known to be associated with a particular gene, which have previously been shown to be associated with a particular trait. Thus, in the present situation, taking the CKM, SCN4α, or LDHα gene, it would be possible, at least in the short term, to select for pigs likely to produce better meat quality, heavy muscling, and/or reduced likelihood of skeletal muscle cramping disease, or alternatively against pigs likely to produce poorer meat quality, heavy muscling, and/or skeletal muscle cramping disease, indirectly, by selecting for certain alleles of a CKM, SCN4α, or LDHα associated marker through the selection of specific alleles of alternative chromosome markers.

As used herein, often the designation of a particular polymorphism is made by the name of a particular restriction enzyme. This is not intended to imply that the only way that the site can be identified is by the use of that restriction enzyme. There are numerous databases and resources available to those of skill in the art to identify other restriction enzymes which can be used to identify a particular polymorphism, for example http://darwin.bio.geneseo.edu which can give restriction enzymes upon analysis of a sequence and the polymorphism to be identified. In fact as disclosed in the teachings herein there are numerous ways of identifying a particular polymorphism or allele with alternate methods which may not even include a restriction enzyme, but which assay for the same genetic or proteomic alternative form.

In yet another embodiment of this invention novel porcine nucleotide sequences have been identified and are disclosed which encode porcine CKM, SCN4α, and LDHα. The cDNA of the porcine CKM, SCN4α, and LDHα gene as well as some intronic DNA sequences are disclosed. These sequences may be used for the design of primers to assay for the SNP's of the invention or for production of recombinant CKM, SCN4α, or LDHα. The invention is intended to include these sequences as well as all conservatively modified variants thereof as well as those sequences which will hybridize under conditions of high stringency to the sequences disclosed. The terms CKM, SCN4α, and LDHα as used herein shall be interpreted to include these conservatively modified variants as well as those hybridized sequences.

The term “conservatively modified variants” applies to both amino acid and nucleic acid sequences. With respect to particular nucleic acid sequences, conservatively modified variants refer to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton, Proteins, W.H. Freeman and Company (1984).

By “encoding” or “encoded”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant, animal, and fungal mitochondria, the bacterium Mycoplasma capricolum, or the ciliate Macronucleus, may be used when the nucleic acid is expressed therein.

The term “stringent conditions” or “stringent hybridization conditions” includes reference to conditions under which a probe will hybridize to its target sequence, to a detectably greater degree than to other sequences (e.g., at least 2-fold over background). Stringent conditions are sequence-dependent and be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 50° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of the complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acids Probes, Part I, Chapter 2, Ausubel, et al., Eds., Greene Publishing and Wiley-Interscience, New York (1995).

### BRIEF DESCRIPTION OF THE DRAWINGS

FIG. 1 shows the results of three haplotypes used to calculate haplotype substitution effects.

### DETAILED DESCRIPTION OF THE PREFERRED EMBODIMENT

The present invention relates to methods that reveal the presence or absence of polymorphisms in the CKM, SCN4α, and LDHα gene. The presence or absence of one or more polymorphisms in these genes has been found to be correlated with meat quality, heavy muscling, and/or skeletal muscle cramping disease in animals.

The creatine kinase-muscle gene encodes a cytoplasmic protein important for energy transduction (ATP+creatine=ADP+phosphocreatine) in such a particularly demanding tissue as skeletal muscle. This gene has a physical map location of SSC6.

The creatine kinase/creatine phosphate system is an energy generating system operative predominantly in the brain, muscle, heart, retina, adipose tissue and the kidney. Wallimann et al., Biochem. J., 281: 21-401 (1992). Creatine kinase is a phosphotransferase, which catalyzes reversibly at localized intracellular sites the transfer of a phosphoryl group from creatine phosphate to ADP to generate ATP which is the main source of energy in the cell. CK plays a key role in the energy homeostasis of cells with intermittently high, fluctuating energy requirements, like neurons, and photoreceptor, and muscle cells. CK is expressed in a tissue specific manner: CK-M (muscle form) and CK-B (brain form). CK is localized in discrete cellular compartments coupled functionally to sites of energy production (glycolysis and mitochondria) or energy consumption (acto-myosin ATPase and Ca⁺⁺-ATPase).

The sodium channel, voltage gated, type IV, alpha (SCN4α) gene encodes an integral membrane protein in skeletal muscle that mediates voltage dependent Na⁺ permeability of excitable membranes which control the excitation-contraction. It has been proposed as a porcine stress syndrome candidate. Mutations in SCN4α in humans and horses cause hyperkalemic period paralysis (HYPP), a disease characterized by hyperexcitability with stiff, cramping muscles. In horses, HYPP appeared among horses selected for heavy muscling. The SCN4α gene is expected to be located on porcine chromosome 12p which is of meat quality interest. Genomic PCR products that have been analyzed include a 563 bp PCR product from SCN4α exon 1-3 (356 bp exonic, 207 bp intronic), and were directly sequenced in several breeds and many potentially useful SNPs have been identified. One SNP affects the predicted amino acid translation in exon 2 (Val to Ile). The coding region was searched for mutations that may be responsible for muscle tremor and extreme heavy muscling in a halothane negative pig. The coding region from a suspect pig and a control pig were amplified by RT-PCR and sequenced from seven overlapping cDNA products. The complete coding region including parts of 5′ UTR and 3′UTR (a total of 6279 bp) was sequenced from each of these pigs. Mutations in additional suspect pigs (another cramping, halothan-negative pig as well as Randy Schmidt's littermates (2) and mothers (2) to stress pigs) were searched for in the largest (about 1232 bp) and perhaps most interesting exon 24 (where some human muscle disease mutations and the equine HYPP mutations are located) amplified from genomic DNA. Altogether a large number of SNPs have been identified in the porcine SCN4α gene, three of which correspond to amino acid changes. PCR-RFLPs (PstI, SalI, and BsrI) have been designed for these three. These SNPs have been associated with meat quality from our association studies.

Lactate dehydrogenase alpha converts lactate to pyruvate in the final step of anaerobic glycolysis. Lactate dehydrogenase (LDH; EC 1.1.127) catalyzes the interconversion of lactate and pyruvate with nicotinamide adenine dinucleotide (NAD⁺) as coenzyme (Everse, J., and N. O. Kaplan. 1973. Lactate dehydrogenase: structure and function. Adv. Enzymol. 28: 61-133). In vertebrates there are three different subunits of LDH isozyes: LDH-A (muscle), LDH-B (heart), and LDA-C (testis). (Market, C. L., Shaklee, J. B. & Whitt, G. S. (1975) Science 189, 102-114).

According to the invention, applicants have identified several different alleles of the CKM, SCN4α, and LDHα gene which are correlated with improved meat quality, heavy muscling, and/or likelihood of skeletal muscle cramping disease in animals.

It is to be understood that the inventions disclosed herein are not limited to the particular methodology, protocols, animal species or genera described herein, and as such may vary. It is also to be understood that the terminology used herein is for the purpose of describing particular embodiments only, and not intended to limit the scope of the present invention which will be limited only by the appended claims.

For the purposes of the present invention, the following terms have been defined as follows:

As used herein “allele” means an alternative form of a genetic locus.

As used herein, “locus” refers to a nucleic acid region where a polymorphic nucleic acid resides.

As used herein, a “probe nucleic acid” is an RNA or DNA or analogue thereof. The probe may be of any length. Typical probes include PCR primers, PCR amplicons, and cloned genomic nucleic acids encoding genetic locus of interest.

As used herein “genetic marker” means any morphological, biochemical, or nucleic acid-based phenotypic difference which reveals a DNA polymorphism. Examples of genetic markers include but are not limited to RFLPs, RAPDs, and AFLPs.

As used herein “favorable meat quality and/or muscle growth” refers to favorable meat quality, heavy muscling, and/or likelihood of skeletal muscle cramping disease. It means a significant increase or decrease (improvement) in one of many measurable meat quality or muscle growth traits (heavy muscling and/or skeletal muscle cramping disease) above the mean of a given population, so that this information can be used to achieve a uniform population which is optimized for meat quality and/ or muscle growth, this may include an increase in some traits or a decrease in others depending on the desired characteristics.

As used herein “genotyping” means the process of determining the genetic composition of individuals using candidate genes and genetic markers.

As used herein “genotype” means the genetic constitution of an organism, as distinguished from its physical appearance (its phenotype).

According to the invention, the association of these polymorphisms with theses trait(s) enables genetic markers to be identified for specific breeds or genetic lines or animals, with meat quality, heavy muscling, and/or skeletal muscle cramping disease early in the animal's life.

One of the single nucleotide polymorphisms identified according to the invention represents a single nucleotide change from a C (allele 1) to a T (allele 2), located in 5′ UTR of the CKM gene (SEQ ID NO:1). A test for this polymorphism was developed using the restriction enzyme MspAlI.

Yet another embodiment of the invention represents a single nucleotide polymorphism identified by a change from a G (allele 1) to a T (allele 2) in the CKM gene (SEQ ID NO:2). A test for this polymorphism was developed using the restriction enzyme BamHI.

According to another embodiment of the present invention is a 9 base pair (bp) insertion/deletion in exon 2 (SEQ ID NO:2) of the CKM gene. The observed alleles are a-TGAGCTTCC-nucleotide sequence in allele 1 that is not present in allele 2.

Further haplotype analysis was conducted to identify favorable combinations of the markers identified in CKM (see Example 10).

Yet another embodiment is a single nucleotide polymorphism found in the porcine SCN4α gene represented by the following changes: (a) a C (allele 1) to a G (allele 2) in exon 24 (SEQ ID NO:3); (b) a G/A in exon 11 (SEQ ID NO:4); or a G/A in exon 2 (SEQ ID NO:5). Tests for these polymorphisms were developed using restrictions enzymes BsrI in (a), PstI in (b) and SalI in (c). This gene has a physical map location of SSC12 (⅔) p13-p11.

Another single nucleotide polymorphism identified according to the present invention is a silent mutation in exon 5 (SEQ ID NO:6) of the porcine LDH-α gene, characterized by a polymorphic base R, wherein R is a G or an A. A test for this polymorphism was developed using the restriction enzyme AciI.

The invention thus relates to genetic markers for economically valuable traits in animals. The markers represent alleles that are associated significantly with meat quality, heavy muscling, and/or skeletal muscle cramping disease trait and thus provides a method of genotyping animals to determine those more likely to produce meat quality, heavy muscling, and/or skeletal muscle cramping disease (levels of one or all of these) when bred by identifying the presence or absence of a polymorphism in the CKM, SCN4α, or LDHα gene that is so correlated with these traits.

Thus, the invention relates to genetic markers and methods of identifying those markers in an animal of a particular animal, breed, strain, population, or group, whereby the animal is more likely to yield meat of meat quality, heavy muscling, and/or skeletal muscle cramping disease.

Any method of identifying the presence or absence of these markers may be used, including, for example, single-strand conformation polymorphism (SSCP) analysis, base excision sequence scanning (BESS), RFLP analysis, heteroduplex analysis, denaturing gradient gel electrophoresis, and temperature gradient electrophoresis, allelic PCR, ligase chain reaction direct sequencing, mini sequencing, nucleic acid hybridization, micro-array-type detection of the CKM, SCN4α, or LDHα gene, or other linked sequences of the CKM, SCN4α, or LDHα gene. Also within the scope of the invention includes assaying for protein conformational or sequences changes which occur in the presence of this polymorphism. The polymorphism may or may not be the causative mutation but will be indicative of the presence of this change and one may assay for the genetic or protein bases for the phenotypic difference.

The following is a general overview of techniques which can be used to assay for the polymorphisms of the invention.

In the present invention, a sample of genetic material is obtained from an animal. Samples can be obtained from blood, tissue, semen, etc. Generally, peripheral blood cells are used as the source, and the genetic material is DNA. A sufficient amount of cells are obtained to provide a sufficient amount of DNA for analysis. This amount will be known or readily determinable by those skilled in the art. The DNA is isolated from the blood cells by techniques known to those skilled in the art.

Isolation and Amplification of Nucleic Acid

Samples of genomic DNA are isolated from any convenient source including saliva, buccal cells, hair roots, blood, cord blood, amniotic fluid, interstitial fluid, peritoneal fluid, chorionic villus, and any other suitable cell or tissue sample with intact interphase nuclei or metaphase cells. The cells can be obtained from solid tissue as from a fresh or preserved organ or from a tissue sample or biopsy. The sample can contain compounds which are not naturally intermixed with the biological material such as preservatives, anticoagulants, buffers, fixatives, nutrients, antibiotics, or the like.

Methods for isolation of genomic DNA from these various sources are described in, for example, Kirby, DNA Fingerprinting, An Introduction, W.H. Freeman & Co. New York (1992). Genomic DNA can also be isolated from cultured primary or secondary cell cultures or from transformed cell lines derived from any of the aforementioned tissue samples.

Samples of animal RNA can also be used. RNA can be isolated from tissues expressing the gene as described in Sambrook et al., supra. RNA can be total cellular RNA, mRNA, poly A+ RNA, or any combination thereof. For best results, the RNA is purified, but can also be unpurified cytoplasmic RNA. RNA can be reverse transcribed to form DNA which is then used as the amplification template, such that the PCR indirectly amplifies a specific population of RNA transcripts. See, e.g., Sambrook, supra, Kawasaki et al., Chapter 8 in PCR Technology, (1992) supra, and Berg et al., Hum. Genet. 85:655-658 (1990).

PCR Amplification

The most common means for amplification is polymerase chain reaction (PCR), as described in U.S. Pat. Nos. 4,683,195; 4,683,202; and 4,965,188 each of which is hereby incorporated by reference. If PCR is used to amplify the target regions in blood cells, heparinized whole blood should be drawn in a sealed vacuum tube kept separated from other samples and handled with clean gloves. For best results, blood should be processed immediately after collection; if this is impossible, it should be kept in a sealed container at 4° C. until use. Cells in other physiological fluids may also be assayed. When using any of these fluids, the cells in the fluid should be separated from the fluid component by centrifugation.

Tissues should be roughly minced using a sterile, disposable scalpel and a sterile needle (or two scalpels) in a 5 mm Petri dish. Procedures for removing paraffin from tissue sections are described in a variety of specialized handbooks well known to those skilled in the art.

To amplify a target nucleic acid sequence in a sample by PCR, the sequence must be accessible to the components of the amplification system. One method of isolating target DNA is crude extraction which is useful for relatively large samples. Briefly, mononuclear cells from samples of blood, amniocytes from amniotic fluid, cultured chorionic villus cells, or the like are isolated by layering on a sterile Ficoll-Hypaque gradient by standard procedures. Interphase cells are collected and washed three times in sterile phosphate buffered saline before DNA extraction. If testing DNA from peripheral blood lymphocytes, an osmotic shock (treatment of the pellet for 10 sec with distilled water) is suggested, followed by two additional washings if residual red blood cells are visible following the initial washes. This will prevent the inhibitory effect of the heme group carried by hemoglobin on the PCR reaction. If PCR testing is not performed immediately after sample collection, aliquots of 10⁶ cells can be pelleted in sterile Eppendorf tubes and the dry pellet frozen at −20° C. until use.

The cells are resuspended (10⁶ nucleated cells per 100 μl) in a buffer of 50 mM Tris-HCl (pH 8.3), 50 mM KCl 1.5 mM MgCl₂, 0.5% Tween 20, and 0.5% NP40 supplemented with 100 μg/ml of proteinase K. After incubating at 56° C. for 2 hr. the cells are heated to 95° C. for 10 min to inactivate the proteinase K and immediately moved to wet ice (snap-cool). If gross aggregates are present, another cycle of digestion in the same buffer should be undertaken. Ten μl of this extract is used for amplification.

When extracting DNA from tissues, e.g., chorionic villus cells or confluent cultured cells, the amount of the above mentioned buffer with proteinase K may vary according to the size of the tissue sample. The extract is incubated for 4-10 hrs at 50°-60° C. and then at 95° C. for 10 minutes to inactivate the proteinase. During longer incubations, fresh proteinase K should be added after about 4 hr at the original concentration.

When the sample contains a small number of cells, extraction may be accomplished by methods as described in Higuchi, “Simple and Rapid Preparation of Samples for PCR”, in PCR Technology, Ehrlich, H. A. (ed.), Stockton Press, New York, which is incorporated herein by reference. PCR can be employed to amplify target regions in very small numbers of cells (1000-5000) derived from individual colonies from bone marrow and peripheral blood cultures. The cells in the sample are suspended in 20 μl of PCR lysis buffer (10 mM Tris-HCl (pH 8.3), 50 mM KCl, 2.5 mM MgCl₂, 0.1 mg/ml gelatin, 0.45% NP40, 0.45% Tween 20) and frozen until use. When PCR is to be performed, 0.6 μl of proteinase K (2 mg/ml) is added to the cells in the PCR lysis buffer. The sample is then heated to about 60° C. and incubated for 1 hr. Digestion is stopped through inactivation of the proteinase K by heating the samples to 95° C. for 10 min and then cooling on ice.

A relatively easy procedure for extracting DNA for PCR is a salting out procedure adapted from the method described by Miller et al., Nucleic Acids Res. 16:1215 (1988), which is incorporated herein by reference. Mononuclear cells are separated on a Ficoll-Hypaque gradient. The cells are resuspended in 3 ml of lysis buffer (10 mM Tris-HCl, 400 mM NaCl, 2 mM Na₂ EDTA, pH 8.2). Fifty μl of a 20 mg/ml solution of proteinase K and 150 μl of a 20% SDS solution are added to the cells and then incubated at 37° C. overnight. Rocking the tubes during incubation will improve the digestion of the sample. If the proteinase K digestion is incomplete after overnight incubation (fragments are still visible), an additional 50 μl of the 20 mg/ml proteinase K solution is mixed in the solution and incubated for another night at 37° C. on a gently rocking or rotating platform. Following adequate digestion, one ml of a 6M NaCl solution is added to the sample and vigorously mixed. The resulting solution is centrifuged for 15 minutes at 3000 rpm. The pellet contains the precipitated cellular proteins, while the supernatant contains the DNA. The supernatant is removed to a 15 ml tube that contains 4 ml of isopropanol. The contents of the tube are mixed gently until the water and the alcohol phases have mixed and a white DNA precipitate has formed. The DNA precipitate is removed and dipped in a solution of 70% ethanol and gently mixed. The DNA precipitate is removed from the ethanol and air-dried. The precipitate is placed in distilled water and dissolved.

Kits for the extraction of high-molecular weight DNA for PCR include a Genomic Isolation Kit A.S.A.P. (Boehringer Mannheim, Indianapolis, Ind.), Genomic DNA Isolation System (GIBCO BRL, Gaithersburg, Md.), Elu-Quik DNA Purification Kit (Schleicher & Schuell, Keene, N.H.), DNA Extraction Kit (Stratagene, LaJolla, Calif.), TurboGen Isolation Kit (Invitrogen, San Diego, Calif.), and the like. Use of these kits according to the manufacturer's instructions is generally acceptable for purification of DNA prior to practicing the methods of the present invention.

The concentration and purity of the extracted DNA can be determined by spectrophotometric analysis of the absorbance of a diluted aliquot at 260 nm and 280 nm. After extraction of the DNA, PCR amplification may proceed. The first step of each cycle of the PCR involves the separation of the nucleic acid duplex formed by the primer extension. Once the strands are separated, the next step in PCR involves hybridizing the separated strands with primers that flank the target sequence. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid.

In a particularly useful embodiment of PCR amplification, strand separation is achieved by heating the reaction to a sufficiently high temperature for a sufficient time to cause the denaturation of the duplex but not to cause an irreversible denaturation of the polymerase (see U.S. Pat. No. 4,965,188, incorporated herein by reference). Typical heat denaturation involves temperatures ranging from about 80° C. to 105° C. for times ranging from seconds to minutes. Strand separation, however, can be accomplished by any suitable denaturing method including physical, chemical, or enzymatic means. Strand separation may be induced by a helicase, for example, or an enzyme capable of exhibiting helicase activity. For example, the enzyme RecA has helicase activity in the presence of ATP. The reaction conditions suitable for strand separation by helicases are known in the art (see Kuhn Hoffman-Berling, 1978, CSH-Quantitative Biology, 43:63-67; and Radding, 1982, Ann. Rev. Genetics 16:405-436, each of which is incorporated herein by reference).

Template-dependent extension of primers in PCR is catalyzed by a polymerizing agent in the presence of adequate amounts of four deoxyribonucleotide triphosphates (typically dATP, dGTP, dCTP, and dTTP) in a reaction medium comprised of the appropriate salts, metal cations, and pH buffering systems. Suitable polymerizing agents are enzymes known to catalyze template-dependent DNA synthesis. In some cases, the target regions may encode at least a portion of a protein expressed by the cell. In this instance, mRNA may be used for amplification of the target region. Alternatively, PCR can be used to generate a cDNA library from RNA for further amplification, the initial template for primer extension is RNA. Polymerizing agents suitable for synthesizing a complementary, copy-DNA (cDNA) sequence from the RNA template are reverse transcriptase (RT), such as avian myeloblastosis virus RT, Moloney murine leukemia virus RT, or Thermus thermophilus (Tth) DNA polymerase, a thermo stable DNA polymerase with reverse transcriptase activity marketed by Perkin Elmer Cetus, Inc. Typically, the genomic RNA template is heat degraded during the first denaturation step after the initial reverse transcription step leaving only DNA template. Suitable polymerases for use with a DNA template include, for example, E. coli DNA polymerase I or its Klenow fragment, T4 DNA polymerase, Tth polymerase, and Taq polymerase, a heat-stable DNA polymerase isolated from Thermus aquaticus and commercially available from Perkin Elmer Cetus, Inc. The latter enzyme is widely used in the amplification and sequencing of nucleic acids. The reaction conditions for using Taq polymerase are known in the art and are described in Gelfand, 1989, PCR Technology, supra.

Allele Specific PCR

Allele-specific PCR differentiates between target regions differing in the presence of absence of a variation or polymorphism. PCR amplification primers are chosen which bind only to certain alleles of the target sequence. This method is described by Gibbs, Nucleic Acid Res. 17:12427-2448 (1989).

Allele Specific Oligonucleotide Screening Methods

Further diagnostic screening methods employ the allele-specific oligonucleotide (ASO) screening methods, as described by Saiki et al., Nature 324:163-166 (1986). Oligonucleotides with one or more base pair mismatches are generated for any particular allele. ASO screening methods detect mismatches between variant target genomic or PCR amplified DNA and non-mutant oligonucleotides, showing decreased binding of the oligonucleotide relative to a mutant oligonucleotide. Oligonucleotide probes can be designed so that under low stringency, they will bind to both polymorphic forms of the allele, but at high stringency, bind to the allele to which they correspond. Alternatively, stringency conditions can be devised in which an essentially binary response is obtained, i.e., an ASO corresponding to a variant form of the target gene will hybridize to that allele, and not to the wild-type allele.

Ligase Mediated Allele Detection Method

Target regions of a test subject's DNA can be compared with target regions in unaffected and affected family members by ligase-mediated allele detection. See Landegren et al., Science 241:107-1080 (1988). Ligase may also be used to detect point mutations in the ligation amplification reaction described in Wu et al., Genomics 4:560-569 (1989). The ligation amplification reaction (LAR) utilizes amplification of specific DNA sequence using sequential rounds of template dependent ligation as described in Wu, supra, and Barany, Proc. Nat. Acad. Sci. 88:189-193 (1990).

Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. DNA molecules melt in segments, termed melting domains, under conditions of increased temperature or denaturation. Each melting domain melts cooperatively at a distinct, base-specific melting temperature (Tₘ). Melting domains are at least 20 base pairs in length, and may be up to several hundred base pairs in length.

Differentiation between alleles based on sequence specific melting domain differences can be assessed using polyacrylamide gel electrophoresis, as described in Chapter 7 of Erlich, ed., PCR Technology, “Principles and Applications for DNA Amplification”, W.H. Freeman and Co., New York (1992), the contents of which are hereby incorporated by reference.

Generally, a target region to be analyzed by denaturing gradient gel electrophoresis is amplified using PCR primers flanking the target region. The amplified PCR product is applied to a polyacrylamide gel with a linear denaturing gradient as described in Myers et al., Meth. Enzymol. 155:501-527 (1986), and Myers et al., in Genomic Analysis, A Practical Approach, K. Davies Ed. IRL Press Limited, Oxford, pp. 95-139 (1988), the contents of which are hereby incorporated by reference. The electrophoresis system is maintained at a temperature slightly below the Tm of the melting domains of the target sequences.

In an alternative method of denaturing gradient gel electrophoresis, the target sequences may be initially attached to a stretch of GC nucleotides, termed a GC clamp, as described in Chapter 7 of Erlich, supra. Preferably, at least 80% of the nucleotides in the GC clamp are either guanine or cytosine. Preferably, the GC clamp is at least 30 bases long. This method is particularly suited to target sequences with high Tₘ's.

Generally, the target region is amplified by the polymerase chain reaction as described above. One of the oligonucleotide PCR primers carries at its 5′ end, the GC clamp region, at least 30 bases of the GC rich sequence, which is incorporated into the 5′ end of the target region during amplification. The resulting amplified target region is run on an electrophoresis gel under denaturing gradient conditions as described above. DNA fragments differing by a single base change will migrate through the gel to different positions, which may be visualized by ethidium bromide staining.

Temperature Gradient Gel Electrophoresis

Temperature gradient gel electrophoresis (TGGE) is based on the same underlying principles as denaturing gradient gel electrophoresis, except the denaturing gradient is produced by differences in temperature instead of differences in the concentration of a chemical denaturant. Standard TGGE utilizes an electrophoresis apparatus with a temperature gradient running along the electrophoresis path. As samples migrate through a gel with a uniform concentration of a chemical denaturant, they encounter increasing temperatures. An alternative method of TGGE, temporal temperature gradient gel electrophoresis (TTGE or tTGGE) uses a steadily increasing temperature of the entire electrophoresis gel to achieve the same result. As the samples migrate through the gel the temperature of the entire gel increases, leading the samples to encounter increasing temperature as they migrate through the gel. Preparation of samples, including PCR amplification with incorporation of a GC clamp, and visualization of products are the same as for denaturing gradient gel electrophoresis.

Single-Strand Conformation Polymorphism Analysis

Target sequences or alleles at the CKM, SCN4α, and LDHα loci can be differentiated using single-strand conformation polymorphism analysis, which identifies base differences by alteration in electrophoretic migration of single-stranded PCR products, as described in Orita et al., Proc. Nat. Acad. Sci. 85:2766-2770 (1989). Amplified PCR products can be generated as described above, and heated or otherwise denatured, to form single-stranded amplification products. Single-stranded nucleic acids may refold or form secondary structures which are partially dependent on the base sequence. Thus, electrophoretic mobility of single-stranded amplification products can detect base-sequence difference between alleles or target sequences.

Chemical or Enzymatic Cleavage of Mismatches

Differences between target sequences can also be detected by differential chemical cleavage of mismatched base pairs, as described in Grompe et al., Am. J. Hum. Genet. 48:212-222 (1991). In another method, differences between target sequences can be detected by enzymatic cleavage of mismatched base pairs, as described in Nelson et al., Nature Genetics 4:11-18 (1993). Briefly, genetic material from an animal and an affected family member may be used to generate mismatch free heterohybrid DNA duplexes. As used herein, “heterohybrid” means a DNA duplex strand comprising one strand of DNA from one animal, and a second DNA strand from another animal, usually an animal differing in the phenotype for the trait of interest. Positive selection for heterohybrids free of mismatches allows determination of small insertions, deletions or other polymorphisms that may be associated with CKM, SCN4α, and LDHα polymorphisms.

Non-gel Systems

Other possible techniques include non-gel systems such as TAQMAN™ (Perkin Elmer). In this system, oligonucleotide PCR primers are designed that flank the mutation in question and allow PCR amplification of the region. A third oligonucleotide probe is then designed to hybridize to the region containing the base subject to change between different alleles of the gene. This probe is labeled with fluorescent dyes at both the 5′ and 3′ ends. These dyes are chosen such that while in this proximity to each other the fluorescence of one of them is quenched by the other and cannot be detected. Extension by Taq DNA polymerase from the PCR primer positioned 5′ on the template relative to the probe leads to the cleavage of the dye attached to the 5′ end of the annealed probe through the 5′ nuclease activity of the Taq DNA polymerase. This removes the quenching effect allowing detection of the fluorescence from the dye at the 3′ end of the probe. The discrimination between different DNA sequences arises through the fact that if the hybridization of the probe to the template molecule is not complete, i.e., there is a mismatch of some form, the cleavage of the dye does not take place. Thus, only if the nucleotide sequence of the oligonucleotide probe is completely complimentary to the template molecule to which it is bound will quenching be removed. A reaction mix can contain two different probe sequences each designed against different alleles that might be present thus allowing the detection of both alleles in one reaction.

Yet another technique includes an Invader Assay, which includes isothermic amplification that relies on a catalytic release of fluorescence. See Third Wave Technology at www.twt.com.

Non-PCR Based DNA Diagnostics

The identification of a DNA sequence linked to CKM, SCN4α, and LDHα can be made without an amplification step, based on polymorphisms including restriction fragment length polymorphisms in an animal and a family member. Hybridization probes are generally oligonucleotides which bind through complementary base pairing to all or part of a target nucleic acid. Probes typically bind target sequences lacking complete complementarity with the probe sequence depending on the stringency of the hybridization conditions. The probes are preferably labeled directly or indirectly, such that by assaying for the presence or absence of the probe, one can detect the presence or absence of the target sequence. Direct labeling methods include radioisotope labeling, such as with P³² or S³⁵. Indirect labeling methods include fluorescent tags, biotin complexes which may be bound to avidin or streptavidin, or peptide or protein tags. Visual detection methods include photoluminescents, Texas red, rhodamine and its derivatives, red leuco dye and 3,3′,5,5′-tetramethylbenzidine (TMB), fluorescein, and its derivatives, dansyl, umbelliferone and the like or with horse radish peroxidase, alkaline phosphatase and the like.

Hybridization probes include any nucleotide sequence capable of hybridizing to the porcine chromosome where CKM, SCN4α, and LDHα resides, and thus defining a genetic marker linked to CKM, SCN4α, and LDHα, including a restriction fragment length polymorphism, a hypervariable region, repetitive element, or a variable number tandem repeat. Hybridization probes can be any gene or a suitable analog. Further suitable hybridization probes include exon fragments or portions of cDNAs or genes known to map to the relevant region of the chromosome.

Preferred tandem repeat hybridization probes for use according to the present invention are those that recognize a small number of fragments at a specific locus at high stringency hybridization conditions, or that recognize a larger number of fragments at that locus when the stringency conditions are lowered.

One or more additional restriction enzymes and/or probes and/or primers can be used. Additional enzymes, constructed probes, and primers can be determined by routine experimentation by those of ordinary skill in the art and are intended to be within the scope of the invention.

Although the methods described herein may be in terms of the use of a single restriction enzyme and a single set of primers, the methods are not so limited. One or more additional restriction enzymes and/or probes and/or primers can be used, if desired. Indeed, in some situations it may be preferable to use combinations of markers giving specific haplotypes. Additional enzymes, constructed probes and primers can be determined through routine experimentation, combined with the teachings provided and incorporated herein.

According to the invention, polymorphisms in the CKM, SCN4α, and LDHα gene have been identified which have an association with meat quality, heavy muscling, and/or skeletal muscle cramping disease. The presence or absence of the markers, in one embodiment may be assayed by PCR-RFLP analysis using the restriction endonucleases and amplification primers may be designed using analogous human, pig or other CKM, SCN4α, and LDHα sequences due to the high homology in the region surrounding the polymorphisms, or may be designed using known CKM, SCN4α, and LDHα gene sequence data as exemplified in GenBank or even designed from sequences obtained from linkage data from closely surrounding genes based upon the teachings and references herein. The sequences surrounding the polymorphism will facilitate the development of alternate PCR tests in which a primer of about 4-30 contiguous bases taken from the sequence immediately adjacent to the polymorphism is used in connection with a polymerase chain reaction to greatly amplify the region before treatment with the desired restriction enzyme. The primers need not be the exact complement; substantially equivalent sequences are acceptable. The design of primers for amplification by PCR is known to those of skill in the art and is discussed in detail in Ausubel (ed.), Short Protocols in Molecular Biology, 4th Edition, John Wiley and Sons (1999).

The following is a brief description of primer design.

Primer Design Strategy

Increased use of polymerase chain reaction (PCR) methods has stimulated the development of many programs to aid in the design or selection of oligonucleotides used as primers for PCR. Four examples of such programs that are freely available via the Internet are: PRIMER by Mark Daly and Steve Lincoln of the Whitehead Institute (UNIX, VMS, DOS, and Macintosh), Oligonucleotide Selection Program (OSP) by Phil Green and LaDeana Hiller of Washington University in St. Louis (UNIX, VMS, DOS, and Macintosh), PGEN by Yoshi (DOS only), and Amplify by Bill Engels of the University of Wisconsin (Macintosh only). Generally these programs help in the design of PCR primers by searching for bits of known repeated-sequence elements and then optimizing the Tₘ by analyzing the length and GC content of a putative primer. Commercial software is also available and primer selection procedures are rapidly being included in most general sequence analysis packages.

Sequencing and PCR Primers

Designing oligonucleotides for use as either sequencing or PCR primers requires selection of an appropriate sequence that specifically recognizes the target, and then testing the sequence to eliminate the possibility that the oligonucleotide will have a stable secondary structure. Inverted repeats in the sequence can be identified using a repeat-identification or RNA-folding program such as those described above. If a possible stem structure is observed, the sequence of the primer can be shifted a few nucleotides in either direction to minimize the predicted secondary structure. The sequence of the oligonucleotide should also be compared with the sequences of both strands of the appropriate vector and insert DNA. Obviously, a sequencing primer should only have a single match to the target DNA. It is also advisable to exclude primers that have only a single mismatch with an undesired target DNA sequence. For PCR primers used to amplify genomic DNA, the primer sequence should be compared to the sequences in the GenBank database to determine if any significant matches occur. If the oligonucleotide sequence is present in any known DNA sequence or, more importantly, in any known repetitive elements, the primer sequence should be changed.

The methods and materials of the invention may also be used more generally to evaluate pig DNA, genetically type individual pigs, and detect genetic differences in pigs. In particular, a sample of pig genomic DNA may be evaluated by reference to one or more controls to determine if a polymorphism in the CKM, SCN4α, or LDHα gene is present. Preferably, RFLP analysis is performed with respect to the pig CKM, SCN4α, and LDHα gene, and the results are compared with a control. The control is the result of a RFLP analysis of the pig CKM, SCN4α, or LDHα gene of a different pig where the polymorphism(s) of the pig CKM, SCN4α, or LDHα gene is/are known. Similarly, the CKM, SCN4α, or LDHα genotype of a pig may be determined by obtaining a sample of its genomic DNA, conducting RFLP analysis of the CKM, SCN4α, or LDHα gene in the DNA, and comparing the results with a control. Again, the control is the result of RFLP analysis of the CKM, SCN4α, or LDHα gene of a different pig. The results genetically type the pig by specifying the polymorphism(s) in its CKM, SCN4α, or LDHα genes. Finally, genetic differences among pigs can be detected by obtaining samples of the genomic DNA from at least two pigs, identifying the presence or absence of a polymorphism in the CKM, SCN4α, and LDHα gene, and comparing the results.

These assays are useful for identifying the genetic markers relating to meat quality, heavy muscling, and/or skeletal muscle cramping disease, as discussed above, for identifying other polymorphisms in the CKM, SCN4α, or LDHα gene and for the general scientific analysis of pig genotypes and phenotypes.

The examples and methods herein disclose certain gene(s) which has been identified to have a polymorphism(s) which is associated either positively or negatively with a beneficial trait that will have an effect on meat quality, heavy muscling, and/or skeletal muscle cramping disease for animals carrying this polymorphism. The identification of the existence of a polymorphism within a gene is often made by a single base alternative that results in a restriction site in certain allelic forms. A certain allele, however, as demonstrated and discussed herein, may have a number of base changes associated with it that could be assayed for which are indicative of the same polymorphism (allele). Further, other genetic markers or genes may be linked to the polymorphisms disclosed herein so that assays may involve identification of other genes or gene fragments, but which ultimately rely upon genetic characterization of animals for the same polymorphism. Any assay which sorts and identifies animals based upon the allelic differences disclosed herein are intended to be included within the scope of this invention.

One of skill in the art, once a polymorphism has been identified and a correlation to a particular trait established will understand that there are many ways to genotype animals for this polymorphism. The design of such alternative tests merely represents optimization of parameters known to those of skill in the art and is intended to be within the scope of this invention as fully described herein.

The following examples serve to better illustrate the invention described herein and are not intended to limit the invention in any way. Those skilled in the art will recognize that there are several different parameters which may be altered using routine experimentation and are intended to be within the scope of this invention.

### EXAMPLE 1

### Swine Creatine Kinase Muscle (CKM) MspAlI PCR-RFLP Test Protocol

We sequenced full encoding cDNA and part of 5′ UTR and 3′ UTR of porcine creatine kinase muscle gene (CKM). The length of porcine coding cDNA is 1150 bp. A new polymorphism located in 5′ UTR was discovered, and based on this, an MspAlI PCR-RFLP test was developed.

To amplify a CKM AMspAlI Amplimer:

| | |
|---|---|
| 5′ Primer CK522F: | |
| 5′-CAG CCC ATA CAA GGC CAT GG-3′ | (SEQ ID NO:7) |
| 3′ Primer CKPR: | |
| 5′-CTG GCT GGG CTG TGC TGG AATAT CCT | (SEQ ID NO:8) |
| GGA GGC GAC AC-3′ | |

PCR Conditions

1× PCR Reaction:

| | Volume (μl) |
|---|---|
| 10X PCR Buffer B | 1.0 |
| MgCl₂ (15 mM) | 1.0 |
| dNTPs (2 mM) | 1.0 |
| CK522F (10 pmol/μl) | 0.525 |
| CKPR (10 pmol/μl) | 0.525 |
| Promega | |
| Taq Polymerase (5 U/μl) | 0.07 |
| ddWater | 4.88 |
| Total Mix Volume | 9.0 |

Kept PCR reaction mix on ice. Placed PCR 96-well plates or PCR 0.2 ml tubes on ice. Aliquoted 9.0 μl of the mix, and added 1.0 μl of 12.5 ng/μl genomic DNA or 1 μl DNA lysate.

Thermocycling was performed under the following conditions:

| 1. 4 min | 94° C. - 1 cycle |
|---|---|
| 2. 45 sec | 94° C. |
| 3. 45 sec | 62° C. |
| 4. 45 sec | 72° C. |
| 5. Went to step 2 for 35 additional cycles. | |
| 6. 12 min | 72° C. - 1 cycle |

CKM MspAlI Restriction Enzyme Digestion Protocol:

| | 1X |
|---|---|
| | Volume (μl) |
| Buffer C* 10X | 1.0 |
| BSA (10 mg/ml) | 0.1 |
| MspA1I (10 units/μl) | 0.3 |
| ddWater | 5.6 |
| Mix Final Volume | 7.0 |

*Promega

Aliquoted 7 μl of MspAlI mix and added 3 μl PCR product. Incubated at 37° C.

Gel Electrophoresis:

Added 2 μl orange G loading buffer and loaded on a 4.0% Nusieve/Me (3:1) agarose gel. Ran at 150 volts. Products were resolved in about 30 minutes.

| | |
|---|---|
| Fragment sizes for each allele: | Allele 1: 146 bp |
| | Allele 2: 120 bp, 26 bp |
| | Monomorphic fragment: 87 bp |

Shown below is the CKM MspAlI polymorphism surround DNA sequence.

MspAlI-c/t-5′ UTR

| | |
|---|---|
| 5′ . . . CAGCCCATACAAGGCCATGGGGCTGGGC | (SEQ ID NO:1) |
| GCAAGGCACGCCTGGGTTCAGGGTGGGCACGGTGCCC | |
| AGGCAGCGAAGCGAGAGCGCAGCTGCCCTCCACCCCC | |
| CTCCTGGCCAGc/tGGCCCCTCCTGACCAATAGCACA | |
| ACCTGGGCCCCCCCTATAAAAGGCCAGGGCTGCAGTC | |
| CTGTCCTTTGGGTCAGTGTCGCCTCCAGGATACAGAC | |
| GCCCCTTCCAGCACAGCCCAGCCAG . . . 3′ | |

### EXAMPLE 2

### Swine Creatine Kinase Muscle (CKM) BamHI PCR-RFLP Test Protocol

The creatine kinase muscle gene encodes a cytoplasmic protein important for energy transduction (ATP+creatine=ADP+phosphocreatine) in such a particularly demanding tissue as skeletal muscle.

Linkage Map Location:

CKM S0220 rec. fracs.=0.00, lods=22.58
CKM GPI-2 rec. fracs.=0.01, lods=20.48
   This is about ˜1 cM from the CRC locus. The CRC genotype data in the PiGMaP file that was retrieved from ResPig, however, was very poor because it did not show significant linkage to any other marker.
   To amplify a CKM BamHI Amplimer:
Forward Primer (CKF7): 5′-TCT GAC CCA GAG GTG TCA AG-3′ (SEQ ID NO:9)
Reverse Primer (CKMMR): 5′-CAG CCC ACG GTC ATG ATG AA-3′ (SEQ ID NO:10)
   PCR Conditions
Reaction volume: 10 μl
PCR Mix: 1.5 mM MgCl₂
   0.2 mM dNTP
   2.5 pmol of each primer
   0.35 U of Taq polymerase (Promega)
   12.5 ng DNA

Kept PCR reaction mix on ice. Placed PCR 96-well plates or PCR 0.2 ml tubes on ice. Aliquoted 9.0 μl of the mix, and added 1.0 μl 12.5 ng/μl genomic DNA or 1 μl DNA lysate.

Thermocycling was performed under the following conditions using a PTC 100 (MJ Research) program “CKF7R”:

1. 1×(95° C. 1 min)
2. 2×(95° C. 1 min, 57° C. 30 s, 72° C. 30 s)
3. 38×(94° C. 30 s, 57° C. 30 s, 72° C. 30s)

CKM BamHI Restriction Enzyme Digestion Protocol:

10 × NEB buffer

| | | |
|---|---|---|
| BamHI | 1 | μl |
| 100 × BSA | 0.1 | μl |
| ddH₂O | 6.8 | μl |
| BamHI (20 U/μl) | 0.1 | μl (1 U) |
| PCR product | 2 | μl |
| | 10 | μl |

| | |
|---|---|
| Fragment Sizes for each allele: | Allele 1: 193 bp |
| | Allele 2: 105 bp, 88 bp |
| | Monomorphic fragment: bp |
| Gel Detection Method: | 4% Nusieve 3:1 or Metaphor, |
| | appr. 100 Vh |

Porcine CKM Sequence Around the BamHI Single Nucleotide Polymorphism

BamHI-g/t-intronic

| | |
|---|---|
| 5′ . . . TCCATCTGGCTTCACCCTGGACGATGTC | (SEQ ID NO:2) |
| ATCCAGACAGGTGTGGACAATCCAGGTAAGCCTCCTT | |
| GGCGGAGCATCACAGGGCCCGGGGGCTCCGGAAGCTG | |
| CCTGCCGGGCCTTGCGCCCACTCCCTGGGCCTCCATG | |
| TTCCCACCTGTAAAATAGGACCCTACTCACGGGGGCT | |
| GTGGTGAGGACCGAATGAGTTGAGGTGGTGAAGGGCT | |
| TGGGACGGGGCCCGGCACGTGGCAAACCACCCGCTAA | |
| ACATACATGAGCATGAACGGAGGCTCCCCGAGGAAGC | |
| CCTTGATGTTCCCGGCCTCAGTTTCCTCACCTGAAAA | |
| TTGGAACAACATAGGGCTCAGCGCACACAGAGCGGCG | |
| CCTGGCACGCAAGCGAGCTCTTGGATCCTGCCAGGGG | |
| GTGTCATGTTCCAGGCCTCTGTGTCCGCTCCTTTCTC | |
| CAGGGACACCCTGCCAGGGCGAGTGGCACTGGGGCAG | |
| GGGGCCAGGCTCGAGCCTGAGCTTCCGACTCAAGGGG | |
| TGATTGGACGGAGAGGCTCTTTCTCCCACCTGGGAAA | |
| CAAGAGCATCTTTCATGGCTCTTTTTATCTGTGGGGG | |
| CTGATGGTCTAAGGTTCCGAAATTTTTTAGAAGATTC | |
| CACAATTTGGGGACTCTGAAGTAGTTTATGTATATAC | |
| ACACACACACACACACACACA::TATATATA::AAAT | |
| GCTTTTTAGGGCCGCACCTGCGGTATGTGGAGATTCC | |
| CAGGCTAGGGGTCGAATCACAGCTGTACCTGTCAGCC | |
| TACACCACAGCTCACGGCAACGCCAGATCCTTAACCT | |
| GCTGAGCGAGGCCAGGGATCAAACTCATGTCCTCATG | |
| GATCTTAGGCCAGTTTGTTCACCACTGAGCCACGACA | |
| GCAACTCCCGAGGTAGTAATATTTTTAGCCTCCCGCC | |
| CCTCCCCTCCTCACCCTCGACCTTCTCCGTTCTGACC | |
| CAGAGGTGTCAAGTGAACTCCTGTGTGCACGCACACG | |
| TGTGCCCACACAGACACACACACACACACACGTGTGT | |
| GGGCGCAGTCTACACTGGACCCAGGAg/tCCTGGCCA | |
| TTCCGAGCTGCGGACAAGCACCTCTGACCTCAACCCC | |
| CATCCCTGCCAGGTCACCCC . . . 3′ | |

### EXAMPLE 3

### Swine Creatine Kinase Muscle (CKM) 9 bp Insertion/Deletion PCR-RFLP Test Protocol

The gene encodes a cytoplasmic protein important for energy transduction (ATP+creatine=ADP+phosphocreatine) in such a particularly demanding tissue as skeletal muscle.

Linkage Map Location:

CKM S0220 rec. fracs.=0.00, lods=22.58
CKM GPI-2 rec. fracs.=0.01, lods=20.48
   This is about ˜1 cM from the CRC locus. The CRC genotype data in the PiGMaP file that was retrieved from ResPig, however, were very poor because it did not show significant linkage to any other marker.

To amplify a 9 bp insertion/deletion CKM Amplimer

| | |
|---|---|
| Forward Primer (CKF5) | |
| 5′-CGA GGG CTG TTA AAG GCC AAGGCT | (SEQ ID NO:11) |
| CCT TTC TCC AGG GAC AC-3′ | |
| Reverse Primer (CGR6) | |
| 5′-ATC ATG CGC TTC ACC GAC TGGGAG | (SEQ ID NO:12) |
| AAA GAG CCT CTC CGT CC-3′ | |

PCR Conditions
Reaction volume: 10 μl
PCR Mix: 1.5 mM MgCl₂
   0.2 mM dNTP
   2.5 pmol of each primer
   0.35 U of Taq polymerase (Promega)
   12.5 ng DNA
      Thermocycling was performed under the following conditions using a PTC100 (MJ Research) program “CKF5R6”:
1×(95° C. 1 min)
2×(95° C. 1 min, 58° C. 30 s, 72° C. 30 s)
38×(94° C. 30 s, 58° C. 30 s, 72° C. 30 s)

Kept PCR reaction mix on ice. Placed PCR 96-well plates or PCR 0.2 ml tubes on ice. Aliquoted 9.0 μl of the mix, and add 1.0 μl 12.5 ng/μl genomic DNA or 1 μl DNA lysate.

| | |
|---|---|
| PCR Fragment Size: | 110 bp (observed for sequenced allele 1) |
| | 101 bp (observed for sequenced allele 2) |
| | Note: heteroduplexes sometimes appear in |
| | heterozygotes |
| Gel Detection Method: | 4% Nusieve 3:1 or Metaphor, appr. 100 Vh |

Porcine CKM Sequence Around the 9 bp Deletion Polymorphism

9 bp del/ins-TGAGCTTCC-

| | |
|---|---|
| 5′ . . . TCCATCTGGCTTCACCCTGGACGATGTCA | (SEQ ID NO:2) |
| TCCAGACAGGTGTGGACAATCCAGGTAAGCCTCCTTG | |
| GCGGAGCATCACAGGGCCCGGGGGCTCCGGAAGCTGC | |
| CTGCCGGGCCTTGCGCCCACTCCCTGGGCCTCCATGT | |
| TCCCACCTGTAAAATAGGACCCTACTCACGGGGGCTG | |
| TGGTGAGGACCGAATGAGTTGAGGTGGTGAAGGGCTT | |
| GGGACGGGGCCCGGCACGTGGCAAACCACCCGCTAAA | |
| CATACATGAGCATGAACGGAGGCTCCCCGAGGAAGCC | |
| CTTGATGTTCCCGGCCTCAGTTTCCTCACCTGAAAAT | |
| TGGAACAACATAGGGCTCAGCGCACACAGAGCGGCGC | |
| CTGGCACGCAAGCGAGCTCTTGGATCCTGCCAGGGGG | |
| TGTCATGTTCCAGGCCTCTGTGTCCGCTCCTTTCTCC | |
| AGGGACACCCTGCCAGGGCGAGTGGCACTGGGGCAGG | |
| GGGCCAGGCTCGAGCCTGAGCTTCCGACTCAAGGGGT | |
| GATTGGACGGAGAGGCTCTTTCTCCCACCTGGGAAAC | |
| AAGAGCATCTTTCATGGCTCTTTTTATCTGTGGGGGC | |
| TGATGGTCTAAGGTTCCGAAATTTTTTAGAAGATTCC | |
| ACAATTTGGGGACTCTGAAGTAGTTTATGTATATACA | |
| CACACACACACACACACACA::TATATATA::AAATG | |
| CTTTTTAGGGCCGCACCTGCGGTATGTGGAGATTCCC | |
| AGGCTAGGGGTCGAATCACAGCTGTACCTGTCAGCCT | |
| ACACCACAGCTCACGGCAACGCCAGATCCTTAACCTG | |
| CTGAGCGAGGCCAGGGATCAAACTCATGTCCTCATGG | |
| ATCTTAGGCCAGTTTGTTCACCACTGAGCCACGACAG | |
| CAACTCCCGAGGTAGTAATATTTTTAGCCTCCCGCCC | |
| CTCCCCTCCTCACCCTCGACCTTCTCCGTTCTGACCC | |
| AGAGGTGTCAAGTGAACTCCTGTGTGCACGCACACGT | |
| GTGCCCACACAGACACACACACACACACACGTGTGTG | |
| GGCGCAGTCTACACTGGACCCAGGAGCCTGGCCATTC | |
| CGAGCTGCGGACAAGCACCTCTGACCTCAACCCCCAT | |
| CCCTGCCAGGTCACCCC . . . 3′ | |

### EXAMPLE 4

### Swine Sodium Channel, Voltage Gated, Type IV, Alpha (SCN4α) BsrI PCR-RFLP Test Protocol

The swine sodium channel, voltage gated, type IV, alpha (SCN4α) gene encodes an integral membrane protein in skeletal muscle that mediates voltage dependent Na⁺ permeability of excitable membranes which control the excitation-contraction. It has been proposed as a porcine stress syndrome candidate. Mutations in SCN4α in humans and horses cause hyperkalemic periodic paralysis (HYPP), a disease characterized by hyperexcitability with stiff, cramping muscles.

To amplify a 262 bp SCN4α BsrI Amplimer

PCR-RFLP Information

| | |
|---|---|
| Primer Sequences: | |
| Forward (SCF23) | |
| 5′-ACG AGG AGG TGT GCG CCA TCA AG-3′ | (SEQ ID NO:13) |
| Reverse (SCR35) | |
| 5′-ATG AGC ACG AGC CCC ATG GCA G-3′ | (SEQ ID NO:14) |

PCR Conditions:
Reaction volume: 10 μl
PCR Mix: 1.5 mM MgCl₂
0.2 mM dNTP
2.5 pmol of each primer
5% DMSO to be added last of all. Thawed 100% DMSO on heating block, added while mixing with the pipetter to avoid precipitation.
0.35 U of Taq polymerase (Promega)
12.5 ng DNA
   Thermocycling was performed under the following conditions using a PTC100 (MJ Research) program “SCF23R35” (Bugs):
1×(95° C. 1 min)
2×(95° C. 1 min, 64° C. 30 s, 72° C. 30 s)
38×(94° C. 1 min, 64° C. 30 s, 72° C. 30 s)

Kept PCR reaction mix on ice. Placed PCR 96-well plates or PCR 0.2 ml tubes on ice. Aliquoted 9.0 μl of the mix, and added 1.0 μl 12.5 ng/μl genomic DNA or 1 μl DNA lysate.

SCN4α BsrI Restriction Enzyme Digestion Protocol:

| | | |
|---|---|---|
| 10 × NEB 3 | 1 | μl |
| 100 × BSA | 0.1 | μl |
| ddH₂O | 6.7 | μl |
| BsrI (5 U/μl) | 0.2 | μl (1 U) |
| PCR product | 2 | μl |
| | 10 | μl |

Incubated at 37° C.

| | |
|---|---|
| Fragment Sizes for each allele: | Allele 1: 262 bp |
| | Allele 2: 190 bp, 72 bp |
| Gel Detection Method: | 3% Nusieve 3:1 agarose, appr. 150 Vh |

Porcine SCN4α Sequence Around the BsrI Single Nucleotide Polymorphism

BsrI-c/g, exon 24; sequence-exon 24.

| | |
|---|---|
| GCCTCGCCCTCTCCGACCTGATCCAGAAATACTTCGT | (SEQ ID NO:3) |
| GTCCCCCACGCTGTTTCGTGTGATCCGCCTGGCCAGG | |
| ATCGGTCGCGTCCTGCGGCTGATCCGCGGGGCCAAGG | |
| GCATCCGGACGCTGCTCTTTGCCCTCATGATGTCGCT | |
| GCCCGCCCTCTTCAACATCGGCCTGCTCCTCTTCCTG | |
| GTCATGTTCATCTACTCCATCTTCGGCATGTCCAACT | |
| TCGCCTACGTCAAGAAGGAGTCGGGCATCGACGACAT | |
| GTTCAACTTCGAGACCTTCGGCAACAGCATCATCTGC | |
| CTCTTCGAGATCACGACGTCGGCGGGCTGGGACGGGC | |
| TGCTCAACCCCATCCTCAACAGCGGGCCCCCCGACTG | |
| CGACCCCACGCTGGAGAACCCGGGCACCAGCGTCCGG | |
| GGCGACTGCGGCAACCCGTCCATCGGCATCTGCTTCT | |
| TCTGCAGCTACATCATCATCTCCTTCCTCATCGTGGT | |
| CAACATGTACATCGCCATCATCCTGGAGAACTTCAAC | |
| GTGGCCACGGAGGAGAGCAGCGAGCCCCTCGGGGAGG | |
| ACGACTTCGAGATGTTCTACGAGACGTGGGAGAAGTT | |
| CGACCCCGACGCCACGCAGTTCATCGACTACAGCCGC | |
| CTCTCGGACTTCGTGGACACCCTGCAGGAGCCGCTGA | |
| GGATCGCCAAGCCCAACAAGATCAAGCTCATCACCAT | |
| GGACCTGCCCATGGTGCCGGGGGACAAGATCCACTGC | |
| CTGGACATCCTCTTCGCCCTGACCAAGGAGGTCCTGG | |
| GCGACTCTGGGGAGATGGACGCCCTCAAGGAGACCAT | |
| GGAGGAGAAGTTCATGGCTGCCAACCCCTCCAAGGTC | |
| TCCTACGAGCCCATCACCACCACGCTCAAGAGGAAGC | |
| ACGAGGAGGTGTGCGCCATCAAGATCCAGAGGGCCTA | |
| CCGCCGGCACCTGCTCCAGCGCTCCGTGAAGCAGGCG | |
| TCCTACATGTACCGCCAGAGCCACGACGGCGGTGGCG | |
| GCGGGGACGGGGCCCCCGAGAAGGAGGGGCTGATTGC | |
| CGACACCATGAGCAAGATGTACGGCCAGGAGAACGGG | |
| AACAc/gCAGTGCGCAGAGCCAGGGGGAGGCGAGGGG | |
| CTGGACAGGGGCCCCCGAACCTGCCATGGGGCTCGTG | |
| CTCATCAGCCCCTCAGAGGCCGCCCTCCCGCCCACCC | |
| CACCCCTGGGGCAGACTGTGCGCCCCGGGGTCAAAGA | |
| GTCACTTGTCTAG | |

### EXAMPLE 5

### Swine Sodium Channel, Voltage Gated, Type IV, Alpha (SCN4α) PstI PCR-RFLP Test Protocol

This gene encodes an integral membrane protein in skeletal muscle that mediates voltage dependent Na⁺ permeability of excitable membranes which control the excitation-contraction. It has been proposed as a porcine stress syndrome candidate. Mutations in SCN4α in humans and horses cause hyperkalemic periodic paralysis (HYPP), a disease characterized by hyperexcitability with stiff, cramping muscles.

PCR Fragment Size: 236 bp

To amplify a 236 bp SCN4α PstI Amplimer

| | |
|---|---|
| Forward Primer (SCF17) | |
| 5′-GGA AGA GGC CCA CCA GAA G-3′ | (SEQ ID NO:15) |
| Reverse Primer (SCR18) | |
| 5′-CAA GTT GCC CAC GGT GAG G-3′ | (SEQ ID NO:16) |

1× PCR Reaction
Reaction volume: 10 μl
PCR Mix: 1.5 mM MgCl₂

0.2 mM dNTP

2.5 pmol of each primer

0.35 U of Taq polymerase (Promega)

12.5 ng DNA

Kept PCR reaction mix on ice. Placed PCR 96-well plates or PCR 0.2 ml tubes on ice. Aliquoted 9.0 μl of the mix, and add 1.0 μl 12.5 ng/μl genomic DNA or 1 μl DNA lysate.

Thermocycling was performed under the following conditions using either a MJ Research Inc. PTC200 or PTC100 thermocycler:

PTC100 (MJ Research) program “CKF5R6”:
1. 1×(95° C. 1 min)
2. 2×(95° C. 1 min, 58° C. 30 s, 72° C. 30 s)
3. 38×(94° C. 1 min, 58° C. 30 s, 72° C. 30 s)

SCN4α PstI Restriction Enzyme Digestion Protocol:

| | | |
|---|---|---|
| 10 × NEB buffer | 1 | μl |
| 100 × BSA | 0.1 | μl |
| ddH₂O | 6.8 | μl |
| PstI (20 U/μl) | 0.1 | μl (2 U) |
| PCR product | 2 | μl |
| | 10 | μl |

Incubate at 37° C.

| | |
|---|---|
| Fragment Sizes for each allele: | Allele 1: 236 bp |
| | Allele 2: 162 bp, 74 bp |
| Gel Detection Method: | 3% Nusieve 3:1 agarose, appr. 150 Vh |

Shown below is the porcine SCN4α sequence around the PstI single nucleotide polymorphism.

PstI-g/a, exon 11; sequence-exon 11

| | |
|---|---|
| AGCTGGAAGAGGCCCACCAGAAGTGCCCACCGTGGTG | (SEQ ID NO:4) |
| GTACAAGTGCTCCCACAAAGTGCTCATATGGAACTGC | |
| TGCg/aGCCCCTGGATGAAGTTCAAGAACATCATCCA | |
| CCTGATTGTCATGGACCCCTTCGTGGACCTGGGCATC | |
| ACCATCTGCATCGTGCTCAACACCCTCTTCATGGCCA | |
| TGGAGCATTACCCCATGACCGAGGAGTTTGACGCCGT | |
| CCTCACCGTGGGCAACTTG | |

### EXAMPLE 6

### Swine Sodium Channel, Voltage Gated, Type IV, Alpha (SCN4α) SalI PCR-RFLP Test Protocol

The gene encodes an integral membrane protein in skeletal muscle that mediates voltage dependent Na⁺ permeability of excitable membranes which control the excitation-contraction. It has been proposed as a porcine stress syndrome candidate. Mutations in SCN4α in humans and horses cause hyperkalemic periodic paralysis (HYPP), a disease characterized by hyperexcitability with stiff, cramping muscles.

PCR Fragment Size: 153 bp

To amplify a 153 bp SCN4α SalI Amplimer:

| | |
|---|---|
| Forward Primer Sequence: (SCF29) | |
| 5′-CGT CGT CAT CTG TCT GCC TG-3′ | (SEQ ID NO:17) |
| Reverse Primer Sequence: (SCR30) | |
| 5′-ATG GCG CTG CGC CTG TCG A-3′ | (SEQ ID NO:18) |

PCR Conditions
Reaction volume: 10 μl
PCR Mix: 1.5 mM MgCl₂
   0.2 mM dNTP
   2.5 pmol of each primer
   0.35 U of Taq polymerase (Promega)
   12.5 ng DNA

Kept PCR reaction mix on ice. Placed PCR 96-well plates or PCR 0.2 ml tubes on ice. Aliquoted 9.0 μl of the mix, and added 1.0 μl 12.5 ng/μl genomic DNA or 1 μl DNA lysate.

Thermocycling was performed under the following conditions using a PTC100 (MJ Research) program “SCF29R30”:

1×(95° C. 1 min)

2×(95° C. 1 min, 59° C. 30 s, 72° C. 30 s)

38×(94° C. 1 min, 59° C. 30 s, 72° C. 30 s)

SCN4α SalI Restriction Enzyme Digestion Protocol:

| | | |
|---|---|---|
| 10 × D (Promega) | 1 | μl |
| ddH₂O | 6.9 | μl |
| SalI (10 U/μl) | 0.1 | μl (1 U) |
| PCR product | 2 | μl |
| | 10 | μl |
| or | | |
| 10 × NEB buffer SalI | 1 | μl |
| 100 × BSA | 0.1 | μl |
| ddH₂O | 6.9 | μl |
| SalI (20 U/μl) | 0.05 | μl (1 U) |
| PCR product | 2 | μl |
| | 10 | μl |

Incubated at 37° C.

| | |
|---|---|
| Fragment Sizes for each allele: | Allele 1: 153 bp |
| | Allele 2: 134 bp and 19 bp |
| Gel Detection Method: | 3% Nusieve 3:1 agarose, appr. 150 Vh. |

Porcine SCN4α Sequence Around the SalI Single Nucleotide Polymorphism:

SalI-g/a, exon 2; restriction site introduced in
the reverse primer; sequence-between exon 1 and
exon 3.

| | |
|---|---|
| GGCCCCGAGAGCCTGCGCCCCTTCACCCGGGAGTCCC | (SEQ ID NO:5) |
| TGGCTGCCATAGAGCAGCGGGTGGTGGAGGAGGAGGC | |
| CCGGCAGCAGCGGAACAAGCAGATGGAGATCGAGGAG | |
| CCAGAACGGAAGCCTCGCAGCGACCTGGAGGCTGGCA | |
| AAAACCTGCCCCTTATCTATGGGGACCCCCCACCCGA | |
| GGTCATCGGCATCCCTCTGGAGGACCTGGATCCCTAC | |
| TACAGCGACAAGAAGGTCAGGGCCTGGGCGGGTTCCT | |
| CTGTCTGTCTGTCCGTCGTCATCTGTCTGCCTGTCCC | |
| GGGCCTCACAGCTCTCTCCCTGCTTCAGACCTTCATC | |
| GTGCTCAACAAGGGCAAGGCCATCTTCCGCTTCTCTG | |
| CCACGCCTGCTCTCTACGTGCTGAGCCCCTTCAGCg/ | |
| aTCGTCAGGCGCAGCGCCATCAAGGTGCTCATCCACT | |
| CATATCCTGCCAGAGTCGGGCGAGCGCCGGGCTGGGA | |
| AAAGGCAGGGGAGGGGTTTGGGGACAGGCCAAACGGG | |
| GTGCTCTGGCCGGGGAGCACCTCCCTCCCCACCTGCT | |
| CTCTCCCTTTCCTTGACCCCCCCCCCAACGCTGTTCA | |
| GCATGTTCATCATGATCACGATCCTGACCAA | |

### EXAMPLE 7

### Swine LDH-α Exon 5 AciI PCR-RFLP Test Protocol

We detected a SNP in exon 5 of the swine lactate dehydrogenase alpha gene. This is a silent mutation. An AciI PCR-RFLP was subsequently developed for this polymorphism. A 518 bp amplimer is produced with the following PCR protocol. The AciI restriction enzyme digest results in two monomorphic fragments, 16 bp and 8 bp, and the following polymorphic patterns: one 494 bp fragment representing the 11 genotype, three fragments, 494 bp, 415 bp and 79 bp, representing the 12 genotype and two fragments, 415 bp and 79 bp, representing the 22 genotype.

To amplify a 518 bp LDH-α Exon 5/Intron 5 Amplimer

LDH-α Exon 5 Primers:

| | |
|---|---|
| 5′-Primer LDH-α F: | |
| 5′ GTG TGG AGC GGA GTA AAT GT-3′ | (SEQ ID NO:19) |
| 3′-Primer LDH-α R: | |
| 5′ CCC CAG ATC CGA GCC GCG TTG-3′ | (SEQ ID NO:20) |

1X PCR Reaction

| | Volume (μl) |
|---|---|
| 10X PCR Buffer B | 1.0 |
| MgCl₂ (25 mM) | 0.6 |
| dNTPs (2 mM) | 1.0 |
| LDH-α F (5′) (10 pmol/μl) | 0.52 |
| LDH-α R (3′) (10 pmol/μl) | 0.52 |
| Promega | |
| Taq Polymerase (5 U/μl) | 0.1 |
| Water | 5.26 |
| Total Mix Volume | 9.0 |

Kept PCR reaction mix on ice. Placed PCR 96-well plates or PCR 0.2 ml tubes on ice. Aliquoted 9.0 μl of the mix, and added 1.0μl 12.5 ng/μl genomic DNA or 1 μl DNA lysate.

Thermocycling was performed under the following conditions using either a MJ Research Inc. PTC200 or PTC 100 thermocycler:

| 1. | 3 min | 94° C. - 1 cycle |
|---|---|---|
| 2. | 30 sec | 94° C. |
| 3. | 30 sec | 54° C. |
| 4. | 30 sec | 72° C. |
| 5. | Go to step 2 for 35 additional cycles. | |
| 6. | 5 min | 72° C. - 1 cycle |
| 7. | σ5 min | 4° C. - 1 cycle |
| 8. | σ | 25° C. |

LDH-α AciI Restriction Enzyme Digestion Protocol:

1X

| | Volume (μl) |
|---|---|
| NEB* 3 10× Buffer | 1.0 |
| 10X BSA | 1.0 |
| NEB* AciI (10 units/μl) | 0.3 |
| Water | 2.7 |
| Mix Final Volume | 5.0 |

*NEB = New England Biolabs Aliquoted 5 μl AciI mix and added 5 μl PCR product. Incubated at 37° C.

Gel Electrophoresis:

Added 2 μl orange G loading buffer and loaded on a 1.8% Nusieve/Me (3:1) or regular agarose gel. Ran at 150 volts. Products were resolved in about 30 minutes.

| | |
|---|---|
| Fragment Sizes for each allele: | Allele 1: 494 bp |
| | Allele 2: 415 bp and 79 bp |
| | Monomorphic fragments: 16 bp and |
| | 8 bp |

The 16 bp and 8 bp fragments were not visualized on a 1.8% agarose gel.

Intron 4/Exon 5/Intron 5 sequence; Exon 5 Polymorphic base: R (G/A), in bold.

AciI PCR-RFLP:

| | |
|---|---|
| GTGCCTGTGTGGAGCGGAGTAAATGTTGCTGGTGTCT | (SEQ ID NO:6) |
| CCCTGAAGAATCTGCACCCTGAATTAGGCACTGATGC | |
| AGATAAGGAACACTGGAAAGCRGTTCACAAAACAGGT | |
| GGTGGACAGGTAATAGATCTCATAATTTGTAATGTGA | |
| AAGGTTAAAATTTATTATTTTATTTAAAAAACTAAAA | |
| GTTTAATAATATTTGCATTCGATTTACTCTGTCAGAA | |
| AACTTGTTTTCTAAAGCTTTTTAAAATATCATACTAT | |
| AAAAAGGTAAAGGCATTAAAAATTACAGACATTTATA | |
| AATGCTACAGTCTATCTTTATTTGCTGTAATTCTCTA | |
| TAGTATGATAATCTTTGTGTTTGTAATGTAAACTAAT | |
| AAGATAAAAGAGGAGTTCCTGTCGTGGCTCAGTGGAA | |
| ACTATTCTGACTAGTATCCATGAGGATGTAAGTTTGA | |
| TCCCTGACCTTGCTCAGTGGATTAAGGATCAGGCATT | |
| GCTGTGAGCTGTGGTGTAGGTTACAACGCGGCTCGGA | |
| TCTGGGG | |

R = G or A

### EXAMPLE 8

Malek et al. (2001) Mammalian Genome, 12:637-645 (in press) revealed a QTL on SSC 6 for ham pH, based on a Berkshire×Yorkshire (B×Y) three generation reference family. The QTL was mapped on the B×Y map in a region where the CKM gene should located. For these reasons we considered CKM an interesting candidate gene for that QTL and in general for pork quality. We genotyped the entire B×Y reference family and we mapped the CKM gene on to the B×Y map using the MspAlI polymorphism. The size of effect is presented in Table 1.

TABLE 1
Evidence for significant QTL for ham pH for pig chromosome 6.

| | | Location | Additive | | Dominance | |
|---|---|---|---|---|---|---|
| Trait | F-Value^{a} | (cM) | Effect^{b} | (S.E.) | Effect | (S.E.) |
| Ham pH | 6.88 | 53 | −0.032 | 0.013 | 0.052 | 0.019 |

^{a}Chromosome-wise F-statistic thresholds at the 5% level, as determined by permutation test were 5.14.
^{b}Additive (a) and dominance (d) QTL effects correspond to genotype values of +a, d, and −a, respectively, for individuals having inherited two Berkshire alleles, heterozygotes, and individuals with two Yorkshire alleles. Positive additive effects indicate that Berkshire alleles increased the trait, negative that the Berkshire alleles decreased it. Dominance effects are relative to the mean of the two homozygotes.
*Significant at the 5% genome-wise level (F > 8.22)
**Significant at the 1% genome-wise level (F > 9.96)II.

We used this marker to genotype several commercial populations and to estimate the association between the CKM MspAlI alleles and several meat quality and production traits. The frequencies of the genotype classes are presented in Table 2.

TABLE 2
Genotype frequency for the porcine CKM MspA1I PCR-RFLP site

| | | | | Duroc | | Hampshire | |
|---|---|---|---|---|---|---|---|
| Genotype | Berkshire | Duroc 1 | Duroc 2 | Synthetic | Hampshire | Synthetic | Pietrain |
| 1/1 | 9 | 30 | 19 | 0 | 11 | 4 | 0 |
| 1/2 | 57 | 111 | 51 | 15 | 128 | 23 | 3 |
| 2/2 | 42 | 152 | 51 | 108 | 341 | 64 | 85 |
| n | 108 | 293 | 121 | 123 | 480 | 91 | 88 |

Several significant associations were revealed between CKM MspAlI alleles and some of the traits we considered were for example, color, firmness, pH, etc. See Table 3 for a summary.

TABLE 3
Association analysis results (probabilities) between CKM MspA1I alleles and
meat quality traits in several commercial line populations (summary)

| Commercial | | Drip | Loin | Loin | | Muscle | Ham | Ham | Ham | | |
|---|---|---|---|---|---|---|---|---|---|---|---|
| population | Firmness | prct | Minl | Minb | DG | Depth | Minl | Mina | pH | Endwt | Hprorib |
| Berkshire | 0.04 | 0.003 | | | | | | | | | |
| Duroc 1 | | | 0.008 | 0.0001 | 0.05 | 0.03 | | | | | |
| Duroc 2 | | | | | | | | | | | |
| Duroc | | | | | | | 0.02 | 0.05 | | | |
| Synthetic | | | | | | | | | | | |
| Hampshire | | | | | | | | | 0.01 | 0.05 | |
| Pietrain | | | | | | | | | | | |
| Hampshire | | | | | | | | | | | 0.05 |
| Synthetic | | | | | | | | | | | |

Traits:
DG - Lifetime Daily Gain,
Min - Minolta measures of color,
HProRib - Hennessy probe rib

### EXAMPLE 9

### Genotyping Frequencies

Two groups (Line cross A, Linecross B) of commercial slaughter pigs were produced in commercial growing conditions and harvested at a commercial abbatoir. A number of measurements were taken for meat quality (pH, color and drip loss) and carcass characteristics (carcass weight, ham, belly and loin content, loin eye area and depth and lean percentage and fat at the 10^{th} rib). Samples were taken from the pigs for marker genotyping. The two groups represented two different genotypes produced with different sire lines per group as well as different parent sow genotypes per group.
p—probability

The following values apply to all of the tables included in this example.

| Least Square (LS) means significance levels: | α and δ significance levels: |
|---|---|
| a-b p < .3 | a p < .3 |
| c-d p < .1 | b p < .1 |
| e-f p < .05 | c p < .05 |
| g-h p < .01 | d p < .01 |
| i-j p < .005 | e p < .005 |
| k-l p < .001 | f p < .001 |
| m-n p < .0005 | g p < .0005 |
| o-p p < .0001 | h p < .0001 |

1) In CKM, three polymorphisms (markers) are available for this gene and were used to estimate marker effects in slaughter pigs.

a) CKM MspAlI

Genotype Linecross A - n = 548

| A | LSmeans (s.e.) | | | geno | α | | δ | |
|---|---|---|---|---|---|---|---|---|
| Trait | 11 | 12 | 22 | p | trait (s.e.) | p | trait (s.e.) | p |
| pH | 5.99 (0.13) a | 6.14 (0.02) b | 6.14 (0.01) b | .50 | 0.08 (0.07) | a | 0.05 | a |
| 45 min | | | | | | | (0.05) | |
| pH 3 hr | 6.05 (0.12) ac | 5.87 (0.02) b | 5.83 (0.01) a d | .09 | −0.11 (0.06) | 0.08 | −0.05 | a |
| | | | | | | | (0.04) | |
| pH 24 hr | 5.80 (0.09) a | 5.72 (0.01) m | 5.66 (0.01) b n | .0007 | −0.07 (0.05) | a | −0.01 | |
| | | | | | | | (0.03) | |
| Minolta L | 41.22 (1.84) a | 43.10 (0.30) g | 43.98 (0.15) b h | .01 | 1.38 (0.92) | a | 0.33 | |
| | | | | | | | (0.65) | |
| Minolta a | 0.48 (0.56) | 0.83 (0.12) a | 1.01 (0.05) b | .27 | 0.26 (0.28) | | 0.06 | |
| | | | | | | | (0.20) | |
| Minolta b | 8.00 (0.71) a | 8.54 (0.12) i | 8.95 (0.06) b j | .003 | 0.48 (0.35) | a | 0.04 | |
| | | | | | | | (0.25) | |
| Drip_% | 0.72 (1.05) a | 1.76 (0.17) i | 2.33 (0.09) b j | .005 | 0.80 (0.53) | a | 0.16 | |
| | | | | | | | (0.37) | |

Significant effects were observed for measures of pH, color (MinL & b) and drip for Linecross Genotype A. Animals with allele 1 (genotype 11 or 12) are preferred for meat quality (pH and color) and reduced drip loss. (No significant effects were observed in Linecross Genotype B where genotype 22 was absent.)

b) CKM BamHI

Linecross Genotype A - n = 601

| A | geno | |
|---|---|---|
| Trait | 11 | 12 |
| pH | 6.14 (0.01) | 6.14 (0.02) |
| 45 min | | |
| pH 3 hr | 5.83 (0.01) a | 5.87 (0.02) b |
| pH 24 hr | 5.66 (0.01) o | 5.72 (0.01) p |
| minolta L | 43.96 (0.15) e | 43.25 (0.27) f |
| minolta a | 0.98 (0.05) | 0.89 (0.11) |
| minolta b | 8.96 (0.06) o | 8.51 (0.10) p |
| drip_% | 2.35 (0.09) i | 1.82 (0.15) j |

Animals of genotype 22 were absent from the slaughtered pigs, due to the low frequency of this allele in the sire line. Significant effects were observed for two measures of pH (3 hr & 24 hr), color (MinL & b) and drip (no significant effects were observed in Linecross Genotype B where genotype 22 was also absent and there was also a lower frequency of genotype 12).

c) CKM 9 bp Insertion/Deletion

Linecross Genotype A - n = 604

| A | Lsmeans (s.e.) | | | geno | α | | δ | |
|---|---|---|---|---|---|---|---|---|
| Trait | 11 | 12 | 22 | p | trait (s.e.) | p | trait (s.e.) | p |
| carc. wt | 196.9 | 193.3 | 193.0 | .26 | −1.93 | a | −1.10 | a |
| | (2.25) a | (0.93) b | (0.85) b | | (1.18) | | (0.99) | |
| ham_% | 11.71 | 11.80 | 11.90 | .03 | 0.09 | 0.04 | −0.00 | |
| | (0.09) e | (0.04) e | (0.03) f | | (0.05) | | (0.04) | |
| loin_% | 7.60 | 7.68 | 7.77 | .19 | 0.09 | a | 0.00 | |
| | (0.11) a | (0.05) a | (0.04) b | | (0.06) | | (0.05) | |
| lea | 6.55 | 6.68 | 6.76 | .15 | 0.10 | 0.09 | 0.01 | |
| | (0.12) c | (0.05) a | (0.04) d b | | (0.06) | | (0.05) | |
| loin | 2.51 | 2.53 | 2.55 | .46 | 0.02 | | −0.00 | |
| depth | (0.04) | (0.01) | (0.01) | | (0.02) | | (0.02) | |
| lean_% | 56.07 | 56.23 | 56.42 | .62 | 0.18 | | −0.02 | |
| | (0.46) | (0.19) | (0.17) | | (0.24) | | (0.20) | |
| pH 45 min | 6.11 | 6.14 | 6.14 | .64 | 0.01 | | 0.01 | |
| | (0.03) | (0.01) | (0.01) | | (0.02) | | (0.01) | |
| pH 3 hr | 5.87 | 5.86 | 5.82 | .04 | −0.02 | a | 0.01 | |
| | (0.03) | (0.01) e | (0.01) b f | | (0.02) | | (0.01) | |
| pH 24 hr | 5.72 | 5.69 | 5.66 | .01 | −0.03 | 0.01 | −0.00 | |
| | (0.02) ae | (0.01) be | (0.01) f | | (0.01) | | (0.01) | |
| minolta L | 42.94 | 43.86 | 43.79 | .17 | 0.42 | 0.09 | 0.33 | a |
| | (0.46) c | (0.20) d | (0.19) d | | (0.25) | | (0.21) | |
| minolta a | 0.80 | 0.93 | 1.01 | .50 | 0.10 | | 0.02 | |
| | (0.20) | (0.08) | (0.06) | | (0.10) | | (0.09) | |
| minolta b | 8.41 | 8.80 | 8.95 | .01 | 0.27 | 0.004 | 0.08 | |
| | (0.17) e I | (0.08) f a | (0.07) b j | | (0.09) | | (0.08) | |
| drip_% | 1.80 | 2.18 | 2.32 | .16 | 0.26 | 0.06 | 0.08 | |
| | (0.26) ac | (0.12) b | (0.11) d | | (0.14) | | (0.12) | |

Linecross Genotype B - n = 541

| | | | | | | δ | | |
|---|---|---|---|---|---|---|---|---|
| B | LSmeans (s.e.) | | | geno | α | | trait | |
| Trait | 11 | 12 | 22 | p | trait (s.e.) | p | (s.e.) | p |
| carc. wt | 197.1 (9.87) | 196.4 (1.43) c | 199.5 (0.99) d | .18 | 1.22 | | −1.26 | |
| | | | | | (4.94) | | (3.42) | |
| ham_% | 12.48 (0.28) a | 12.09 (0.04) ba | 12.02 (0.03) b | .09 | −0.23 | a | −0.10 | a |
| | | | | | (0.14) | | (0.10) | |
| loin_% | 8.42 (0.34) a | 8.03 (0.05) bc | 7.92 (0.03) b d | .08 | −0.25 | a | −0.09 | |
| | | | | | (0.17) | | (0.12) | |
| lea | 7.89 (0.38) ce | 7.21 (0.06) de | 7.07 (0.04) f | .01 | −0.41 | 0.03 | −0.18 | a |
| | | | | | (0.19) | | (0.13) | |
| loin | 2.77 (0.11) a | 2.66 (0.02) e | 2.62 (0.01) b f | .04 | −0.07 | a | −0.02 | |
| depth | | | | | (0.05) | | (0.04) | |
| lean_% | 58.39 (1.39) a | 56.94 (0.20) c | 56.51 (0.14) b d | .09 | −0.94 | a | −0.34 | |
| | | | | | (0.70) | | (0.48) | |
| 10th rib | 0.81 (0.10) a | 0.92 (0.01) bc | 0.95 (0.01) b d | .07 | 0.07 | a | 0.02 | |
| | | | | | (0.05) | | (0.04) | |

Significant effects are seen in both Linecross Genotypes for the carcass composition traits—ham and loin % and related traits such as lea and loin depth. Higher yields are associated with allele 2 in Linecross Genotype A and with allele 1 in Linecross Genotype B. However, allele 2 is also associated with lower meat quality as judged by a higher pH of 24, lighter meat and higher drip loss. These effects on meat quality were not observed in Linecross Genotype B, although there was an effect on fatness measured at the 10th rib in line with the yield of ham and loin joints. Producers and breeders working with Linecross Genotype B will wish to select for allele 1, while those working with Linecross Genotype A will utilize the marker depending on the economic values of the different traits in the markets they are working in.

It will be realized by those skilled in the art that marker haplotypes can be constructed for markers in the CKM gene and these haplotypes used for association analysis and then as tools for marker assisted selection as an alternative to using the individual markers.

2) LDHα (Exon 5 AciI)

Genotype Linecross A - n = 583

| | | | LS | | | | | |
|---|---|---|---|---|---|---|---|---|
| | | | means | | α | | | |
| | | | (s.e.) | geno | Trait | | δ | |
| Trait | 11 | 12 | 22 | p | (s.e.) | p | Trait (s.e.) | p |
| ham_% | 11.88 | 11.88 | 11.77 | .14 | −0.06 | 0.10 | 0.04 | a |
| | (0.05) c | (0.03) c | (0.05) d | | (0.03) | | (0.03) | |
| loin_% | 7.77 | 7.76 | 7.57 | .02 | −0.10 | 0.02 | 0.06 | a |
| | (0.06) e | (0.04) e | (0.06) f | | (0.04) | | (0.04) | |
| lea | 6.77 | 6.72 | 6.58 | .10 | −0.09 | 0.04 | 0.03 | |
| | (0.06) e | (0.05) c | (0.07) f d | | (0.04) | | (0.04) | |
| loin | 2.56 | 2.54 | 2.49 | .04 | −0.03 | 0.02 | 0.01 | |
| depth | (0.02) e | (0.01) e | (0.02) f | | (0.01) | | (0.01) | |
| pH 45 min | 6.16 | 6.14 | 6.10 | .10 | −0.03 | 0.03 | 0.01 | |
| | 0.02 e | (0.01) a | (0.02) f b | | (0.01) | | (0.01) | |
| pH 3 hr | 5.85 | 5.85 | 5.80 | .08 | −0.02 | 0.06 | 0.02 | a |
| | (0.02) c | (0.01) e | (0.02) d f | | (0.01) | | (0.01) | |
| pH 24 hr | 5.66 | 5.69 | 5.66 | .09 | 0.00 | | 0.02 | 0.03 |
| | (0.01) e | (0.01) f a | (0.01) b | | (0.01) | | (0.01) | |
| Minolta L | 44.03 | 43.70 | 43.37 | .23 | −0.33 | 0.09 | −0.00 | |
| | (0.25) ac | (0.19) b | (0.29) d | | (0.19) | | (0.18) | |
| minolta a | 1.03 | 0.98 | 0.83 | .38 | −0.10 | a | 0.03 | |
| | (0.09) a | (0.07) a | (0.11) b | | (0.07) | | (0.06) | |
| minolta b | 8.99 | 8.77 | 8.67 | .07 | −0.16 | 0.03 | −0.04 | |
| | (0.10) c e | (0.07) d | (0.11) f | | (0.07) | | (0.07) | |

Genotype B - n = 508

| | | | | | α | | | |
|---|---|---|---|---|---|---|---|---|
| | Lsmeans (s.e.) | | | geno | trait | | δ | |
| Trait | 11 | 12 | 22 | p | (s.e.) | p | trait (s.e.) | p |
| ham_% | 12.02 | 12.02 | 12.17 | .08 | 0.08 | 0.04 | −0.05 (0.03) | a |
| | (0.04) e | (0.03) e | (0.06) f | | (0.04) | | | |
| loin_% | 7.92 | 7.94 | 8.09 | .15 | 0.09 | 0.05 | −0.04 (0.04) | a |
| | (0.05) c | (0.04) c | (0.08) d | | (0.04) | | | |
| lea | 7.08 | 7.09 | 7.27 | .13 | 0.10 | 0.05 | −0.06 (0.04) | a |
| | (0.05) c | (0.05) c | (0.09) d | | (0.05) | | | |
| loin | 2.62 | 2.63 | 2.68 | .18 | 0.03 | 0.06 | −0.01 (0.01) | |
| depth | (0.01) c | (0.01) a | (0.03) d b | | (0.01) | | | |
| pH | 6.10 | 6.07 | 6.03 | .03 | −0.04 | 0.01 | 0.01 (0.01) | |
| 45 min | (0.01)a e | (0.01) b | (0.03) a f | | (0.01) | | | |
| pH 3 hr | 5.77 | 5.76 | 5.70 | .04 | −0.04 | 0.01 | 0.01 (0.01) | a |
| | (0.01) e | (0.01) e | (0.03) f | | (0.01) | | | |
| pH 24 hr | 5.65 | 5.65 | 5.58 | .01 | −0.03 | 0.007 | 0.02 (0.01) | 0.03 |
| | (0.01) g | (0.01) i | (0.02) h j | | (0.01) | | | |
| minolta L | 44.35 | 44.57 | 45.95 | .007 | 0.80 | 0.002 | −0.39 (0.23) | 0.01 |
| | (0.26) i | (0.24) g | (0.45) j h | | (0.26) | | | |
| minolta a | 1.29 | 1.26 | 1.11 | .50 | −0.09 | a | 0.04 (0.07) | |
| | (0.08) a | (0.07) | (0.14) b | | (0.08) 3 | | | |
| minolta b | 9.21 | 9.37 | 9.64 | .05 | 0.22 | 0.02 | −0.04 (0.08) | |
| | (0.09) a e | (0.08) b | (0.16) a f | | (0.09) | | | |
| drip_% | 3.02 | 2.99 | 4.14 | .001 | 0.56 | 0.0008 | −0.39 (0.15) | 0.009 |
| | (0.17) k | (0.15) k | (0.29) 1 | | (0.16) | | | |

The marker genotype of the slaughter pigs explains a significant amount of variation in many of the traits measured (p<0.10). In Linecross A animals of genotype 22 have a lower yield of ham and loin as well as smaller loins (lea and loin depth) than animals of genotype 11 or 12. In addition, there are some significant effects on pH and color with animals of genotype 22 tending to have meat that is darker (preferred) MinL score. In this case the heterozygote class has the highest (preferred) pH 24; however, it does not result in any difference in drip loss (not significant). Producers may wish to ensure that they rear animals of genotype 11 or 12 if they wish to increase the yield of lean meat. Producers who are only interested in darker meat may wish to select for animals of genotype 22. Likewise the marker can be used to select for breeding stock using the marker according to the preference of their customers for yield of prime cuts or color.

In Linecross B there is again a highly significant effect of marker genotype on these traits. However, in the case of yield of ham etc genotype 22 is the preferred genotype. However, both pH and color are best for genotype 11 and 12, with a highly significant effect being observed for drip loss with genotype 22 being associated with significantly greater loss of drip. In this case producers wishing to select animals with the best meat quality (and lower drip losses) would wish to select against genotype 22, taking into account the lower yield of meat. Producers who are only interested in yield would select animals of genotype 22. Breeders would select animals with marker genotypes according to the economic weighting of the various traits.

3) In SCN4α, three polymorphisms (markers) are available in this gene and were used to estimate marker effects in slaughter pigs.

a) SCN4α BsrI

Linecross Genotype A - n = 595

| A | Lsmeans (s.e.) | | | geno | α | | δ | |
|---|---|---|---|---|---|---|---|---|
| Trait | 11 | 12 | 22 | p | trait (s.e.) | p | trait (s.e.) | p |
| carc. wt | 193.8 | 192.9 | 193.3 | .86 | −0.23 (0.87) | | −0.04 | |
| | (1.33) | (0.85) | (1.18) | | | | (0.79) | |
| ham_% | 11.95 | 11.8 | 11.90 | .02 | −0.02 (0.03) | | −0.09 | 0.006 |
| | (0.05) e | (0.03) fc | (0.05) d | | | | (0.03) | |
| loin_% | 7.81 | 7.67 | 7.76 | .11 | −0.02 (0.04) | | −0.08 | 0.04 |
| | (0.07) c | (0.04) da | (0.06) b | | | | (0.04) | |
| lea | 6.84 | 6.64 | 6.74 | .05 | −0.05 (0.05) | a | −0.10 | 0.02 |
| | (0.07) ea | (0.04) fa | (0.06) b | | | | (0.04) | |
| loin | 2.56 | 2.52 | 2.55 | .16 | −0.01 (0.01) | | −0.02 | 0.06 |
| depth | (0.02) c | (0.01)d a | (0.02) b | | | | (0.01) | |
| tr belly % | 10.44 | 10.52 | 10.45 | .16 | 0.01 (0.03) | | 0.05 | 0.06 |
| | (0.04) a | (0.03) b | (0.04) a | | | | (0.03) | |
| lean_% | 56.94 | 56.03 | 56.51 | .01 | −0.21 (0.17) | a | −0.46 | 0.004 |
| | (0.27) ia | (0.17)jc | (0.24) b d | | | | (0.16) | |
| 10^{th} rib | 0.91 | 0.97 | 0.94 | .02 | 0.01 (0.01) | | 0.03 | 0.005 |
| | (0.02) g | (0.01) hc | (0.02) d | | | | (0.01) | |
| pH 45 min | 6.15 | 6.14 | 6.12 | .57 | −0.01 (0.01) | a | 0.00 | |
| | (0.02) a | (0.01) | (0.02) b | | | | (0.01) | |
| pH 3 hr | 5.89 | 5.83 | 5.82 | .004 | −0.04 (0.01) | 0.003 | −0.02 | a |
| | (0.02) iI | (0.01) j | (0.02) j | | | | (0.01) | |
| pH 24 hr | 5.70 | 5.67 | 5.66 | .12 | −0.02 (0.01) | 0.04 | −0.00 | |
| | (0.01) ae | (0.01) b | (0.01) f | | | | (0.01) | |
| minolta L | 43.32 | 43.63 | 44.44 | .006 | 0.56 (0.19) | 0.003 | −0.17 | |
| | (0.27) iI | (0.19) e | (0.26) j f | | | | (0.18) | |
| minolta a | 0.83 | 1.02 | 0.96 | .26 | 0.07 (0.07) | | 0.08 | a |
| | (0.10) a | (0.07) b | (0.09) | | | | (0.06) | |
| minolta b | 8.59 | 8.85 | 9.02 | .01 | 0.21 (0.07) | 0.003 | 0.03 | |
| | (0.10) e iI | (0.07) f a | (0.10) j b | | | | (0.07) | |
| drip_% | 1.95 | 2.24 | 2.40 | .10 | 0.23 (0.11) | 0.04 | 0.04 | |
| | (0.15) ae | (0.11) b | (0.15) f | | | | (0.10) | |

Linecross Genotype B - n = 535

| B | LSmeans (s.e.) | | | | α | | δ | |
|---|---|---|---|---|---|---|---|---|
| Trait | 11 | 12 | 22 | geno p | trait (s.e.) | p | trait (s.e.) | p |
| carc. wt | 189.1 | 197.2 | 201.0 | .01 | 5.95 (2.64) | 0.02 | 1.46 | |
| | (5.16) ae | (1.10) be | (1.26) f | | | | (1.89) | |

In Linecross Genotype A marker genotype is significantly associated with variation in carcass composition and meat quality traits. In the case of the yield of carcass components the genotype 12 is generally unfavorable being associated with lower yields of ham and loin, lower lea, loin depth and lean %, although this genotype has a higher yield of belly. The highest yields (except for belly) are associated with genotype 11. Interestingly a different effect is seen with respect to meat quality, where the effects are more consistent with an additive effect of allele 1 for the favorable scores of higher pH, lower MinoltaL (darker meat) and lower drip loss. The only effect associated with the marker in Linecross Genotype B was for carcass weight where allele 2 is associated with heavier carcasses.

b) SCN4α PstI

Linecross Genotype A - n = 609

| A | LSmeans (s.e.) | | | geno | α | | δ | |
|---|---|---|---|---|---|---|---|---|
| Trait | 11 | 12 | 22 | p | trait (s.e.) | p | trait (s.e.) | p |
| pH | 6.14 (0.01) | 6.15 (0.02) | — | .56 | 0.02 (0.03) | | — | |
| 45 min | | | | | | | | |
| pH 3 hr | 5.84 (0.01) | 5.87 (0.02) | — | .18 | 0.03 (0.02) | a | — | |
| pH 24 hr | 5.67 (0.01) | 5.72 (0.02) | — | .004 | 0.05 (0.02) | 0.004 | — | |
| Minolta L | 43.99 (0.14) | 42.48 (0.34) | — | .0001 | −1.51 (0.36) | 0.0001 | — | |
| Minolta b | 8.89 (0.05) | 8.52 (0.13) | — | .008 | −0.38 (0.14) | 0.008 | — | |
| Drip_% | 2.35 (0.08) | 1.54 (0.19) | — | .0001 | −0.81 (0.21) | 0.0001 | — | |

A highly significant effect of this marker was found for pH 24, Minolta L and drip loss in Linecross Genotype A (no effects were significant for Linecross genotype B), with allele 2 being the preferred allele (no animals of marker genotype 22 were observed).

c) SNC4α SalI

Linecross Genotype A - n = 609

| A | LSmeans (s.e.) | | | geno | α | | δ | |
|---|---|---|---|---|---|---|---|---|
| Trait | 11 | 12 | 22 | p | trait (s.e.) | p | trait (s.e.) | p |
| loin_% | — | 7.67 (0.05) | 7.75 (0.04) | .18 | 0.08 (0.06) | a | — | |
| lea | — | 6.66 (0.05) | 6.74 (0.04) | .23 | 0.08 (0.06) | a | — | |
| loin | — | 2.52 (0.02) | 2.54 (0.01) | .14 | 0.03 (0.02) | a | — | |
| depth | | | | | | | | |
| tr. | — | 10.53 (0.03) | 10.48 (0.03) | .21 | −0.05 (0.04) | a | — | |
| belly % | | | | | | | | |
| lean_% | — | 56.03 (0.20) | 56.47 (0.16) | .07 | 0.44 (0.24) | 0.07 | — | |
| 10^{th} rib | — | 0.97 (0.02) | 0.94 (0.01) | .09 | −0.03 (0.02) | 0.09 | — | |

Linecross Genotype B - n = 548

| B | LSmeans (s.e.) | | | geno | α | | δ | |
|---|---|---|---|---|---|---|---|---|
| Trait | 11 | 12 | 22 | p | trait (s.e.) | p | trait (s.e.) | p |
| pH 3 hr | 5.94 (0.12) a | 5.80 (0.02) bc | 5.75 (0.01) bd | .06 | −0.10 (0.06) | a | −0.03 | |
| | | | | | | | (0.04) | |
| pH 24 hr | 5.67 (0.10) | 5.68 (0.02) e | 5.63 (0.01) f | .05 | −0.02 (0.05) | | 0.02 | |
| | | | | | | | (0.03) | |

Here this marker is associated with different traits between the two genotypes. In Linecross Genotype A genotype 22 is associated with larger and leaner loins and a smaller yield of belly. There were no significant effects on meat quality measures. In Linecross Genotype B, allele 2 is associated with lower pH at 3 and 24 hr although there were no correlated effects on drip loss or color.

It will be realized by those skilled in the art that marker haplotypes can be constructed for markers in the SCN4α gene and these haplotypes used for association analysis and then as tools for marker assisted selection as an alternative to using the individual markers.

### EXAMPLE 10

The three CKM markers can be used to generate marker genotypes and haplotypes for different populations in order to refine marker effects. This was undertaken for two of the CKM markers (the 9 bp insertion/deletion and the MspAlI polymorphism) on a set of breeding lines with carcass and meat quality phenotypes. Three haplotypes could be identified, 1-1, 1-2 and 2-2, the fourth possible haplotype 2-1 was not observed in any of the populations.

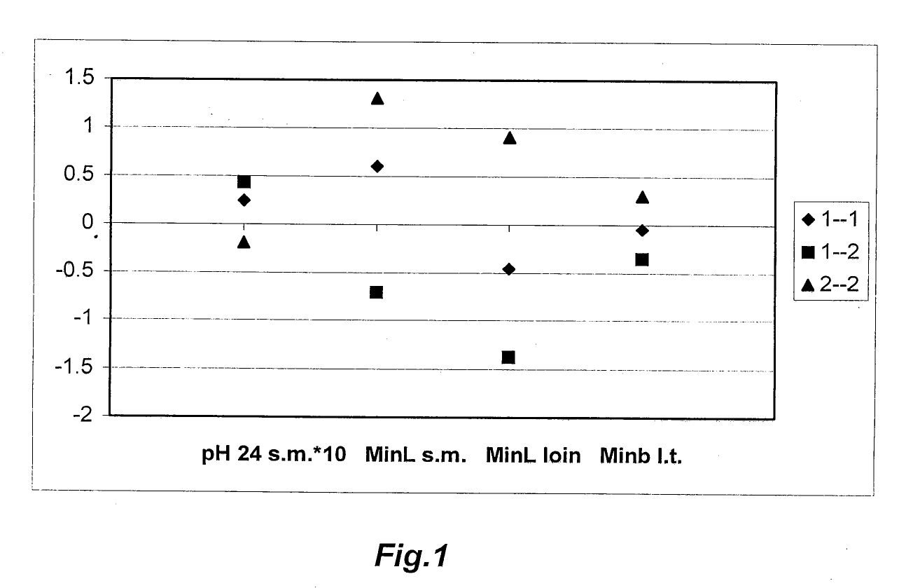

The three haplotypes were then used to calculate haplotype substitution effects (across lines analysis results are presented in FIG. 1 for pH and color traits). It can be seen that haplotype 1-2 was favorable for pH (higher ultimate pH) and color of loin and ham (semi-membranosous) (lower scores equate to darker meat). These effects were approximately 0.07 units for pHu and 2 units for Minolta L between haplotype 1-2 and haplotype 2-2.

Expected differences between homozygotes for haplotype 1-2 or haplotype 2-2 are therefore 0.14 units for pHu and 4 units for Minolta L scores. Neither marker would have shown the full effect (identified by the haplotype analysis) when used on its own. This illustrates the value in some circumstances in combining marker genotypes to generate haplotypes. In some situations, it may be better to utilize all three markers for this purpose.

### EXAMPLE 11

Additional data on the genotypes for different populations is below.

1c) CKM 9 bp Insrertion/Deletion

Genotype Linecross C n = 687

| | LSmeans | | | Geno |
|---|---|---|---|---|
| Trait | 11 | 12 | 22 | P |
| Drip 24 hr | 2.84 | 1.81 | 1.76 | 0.075 |
| Drip 48 hr | 3.79 | 3.17 | 3.08 | 0.035 |
| Sidefat | 2.63 | 2.63 | 2.75 | 0.005 |
| Mean Backfat | 2.48 | 2.45 | 2.46 | NS |

In this independent trial a significant effect of the marker was observed for drip loss at both 24 and 48 hours post mortem (p<0.05 at 48 h). There was also a significant effect on the amount of sidefat on the carcass, however, there was no significant effect on average backfat. In this genotype combination producers wishing to select for animals which would provide lower drip loss post mortem would prefer to have animals of genotype 12 or 22.

2) LDHα

Genotype Linecross C n = 732

| | LSmeans | | | Geno |
|---|---|---|---|---|
| Trait | 11 | 12 | 22 | P |
| Backfat | 3.82 | 3.78 | 3.72 | NS |
| shoulder | | | | |
| Backfat belly | 2.18 | 2.14 | 2.10 | >0.20 |
| Backfat ham | 1.46 | 1.42 | 1.35 | 0.075 |
| Mean Backfat | 2.48 | 2.45 | 2.39 | 0.073 |

In this independent trial a significant effect (p<0.10) of the marker was observed for average carcass backfat and ham backfat. Although the effect was not statistically significant for backfat measured on the shoulder and belly the trend was nevertheless the same, with genotype 22 been the leanest genotype. In this genotype combination producers wishing to provide lean carcasses to the abbatoir would prefer to have animals of genotype 22.
Genotype: Specific Synthetic Line n=5321
Genotypes were generated for many thousands of animals with phenotypic records for average daily feed intake (ADF), backfat, loin depth and pH24hr. In this line genotype 11 had the lowest backfat (approx 0.4 mm less than genotype 22), the highest loin depth (0.6 mm higher than 22), a higher ph 24 hr (0.02 than 22) and had a lower feed intake (0.03 kg less than 22). In this case the effects were estimated using the PEST program which does not provide a significance estimate for large datasets of this type. However, the effects are likely to be statistically significant when based on such a large number of animals.

Those skilled in the art will appreciate that numerous changes and modifications may be preferred embodiments of the invention and that such changes and modification may be made without departing from the spirit of the invention. It is therefore intended that the appended claims cover all such equivalent variations as fall within the true spirit and scope of the invention.

What is claimed is:

1. A method of genetically identifying a marker correlated with favorable muscle growth and/or meat quality comprising:
obtaining a sample of genetic material from said animal;
assaying for the presence of a polymorphic allele in a CKM gene; and
correlating whether a statistically significant association exists between said polymorphic allele and favorable muscle growth and/or favorable meat quality in an animal of a particular breed, strain, population, or group whereby said animal can be characterized for said marker.

2. The method of claim 1 wherein said animal is a pig.

3. The method of claim 1 wherein said assaying is selected from the group consisting of: restriction fragment length polymorphism (RFLP), heteroduplex analysis, single-strand conformational polymorphism (SSCP), denaturing gradient gel electrophoresis (DGGE), single base extension, mass spectrometry, oligo ligation assay (ligase chain reaction), DNA sequencing and temperature gradient gel electrophoresis (TGGE).

4. The method of claim 1 further comprising amplifying an amount of said gene or a portion thereof which contains said polymorphism.

5. The method of claim 4 wherein said amplification includes selecting a forward a reverse primer capable of amplifying a region of said gene which contains at least one polymorphic site.

6. The method of claim 1 wherein said gene is the CKM gene.

7. The method of claim 6 wherein said gene contains a polymorphic MspAlI site.

8. The method of claim 7 wherein said polymorphic site is amplified by primers selected from and based upon SEQ ID NO:7 and SEQ ID NO:8.

9. The method of claim 6 wherein said gene contains a polymorphic BamHI site.

10. The method of claim 9 wherein said polymorphic site is amplified by primers selected from and based upon SEQ ID NO:9 and SEQ ID NO:10.

11. The method of claim 6 wherein said gene contains a polymorphism identified by a 9 base pair insertion/deletion.

12. The method of claim 11 wherein said polymorphism is amplified by primers selected from and based upon SEQ ID NO:11 and SEQ ID NO:12.

13. A method of screening an animal to determine said animal's genetic potential for animal breeding comprising:
obtaining a genetic sample from said animal;
identifying said animal's genotype wherein said genotype has at least one polymorphic site in a gene CKM; and
making genetic assessments based upon the presence of a polymorphism in said gene which is correlated with favorable breeding traits.

14. The method of claim 13 wherein identifying at least one polymorphic site comprises:
amplifying said sample which contains a polymorphism;
generating or destroying a restriction site in said sample;
determining whether a site is cleaved by a specific restriction endonuclease; and
determining whether said sample contains an insertion or deletion, wherein cleavage of a restriction endonuclease site or an insertion or deletion indicates the presence of a polymorphism.

15. The method of claim 13 further comprising running gel electrophoresis to identify polymorphism.

16. The method of claim 13 wherein said genotype is characterized by at least one polymorphism in the CKM gene.


SEQUENCE LISTING (20 sequences)

SEQ ID NO: 1 - DNA, 236 bp, Porcine
  allele (114)..(114): c/t
cagcccatacaaggccatggggctgggcgcaaggcacgcctgggttcagggtgggcacggtgcccaggcagcgaagcgagagcgcagctgccctccacccccctcctggccagcggcccctcctgaccaatagcacaacctgggccccccctataaaaggccagggctgcagtcctgtcctttgggtcagtgtcgcctccaggatacagacgccccttccagcacagcccagccag

SEQ ID NO: 2 - DNA, 1115 bp, Porcine
  misc_feature: 9 bp insertion/deletion tgagcttcc
  allele (1050)..(1050): c/t
tccatctggcttcaccctggacgatgtcatccagacaggtgtggacaatccaggtaagcctccttggcggagcatcacagggcccgggggctccggaagctgcctgccgggccttgcgcccactccctgggcctccatgttcccacctgtaaaataggaccctactcacgggggctgtggtgaggaccgaatgagttgaggtggtgaagggcttgggacggggcccggcacgtggcaaaccacccgctaaacatacatgagcatgaacggaggctccccgaggaagcccttgatgttcccggcctcagtttcctcacctgaaaattggaacaacatagggctcagcgcacacagagcggcgcctggcacgcaagcgagctcttggatcctgccagggggtgtcatgttccaggcctctgtgtccgctcctttctccagggacaccctgccagggcgagtggcactggggcagggggccaggctcgagcctgagcttccgactcaaggggtgattggacggagaggctctttctcccacctgggaaacaagagcatctttcatggctctttttatctgtgggggctgatggtctaaggttccgaaattttttagaagattccacaatttggggactctgaagtagtttatgtatatacacacacacacacacacacacatatatataaaatgctttttagggccgcacctgcggtatgtggagattcccaggctaggggtcgaatcacagctgtacctgtcagcctacaccacagctcacggcaacgccagatccttaacctgctgagcgaggccagggatcaaactcatgtcctcatggatcttaggccagtttgttcaccactgagccacgacagcaactcccgaggtagtaatatttttagcctcccgcccctcccctcctcaccctcgaccttctccgttctgacccagaggtgtcaagtgaactcctgtgtgcacgcacacgtgtgcccacacagacacacacacacacacacgtgtgtgggcgcagtctacactggacccaggagcctggccattccgagctgcggacaagcacctctgacctcaacccccatccctgccaggtcacccc

SEQ ID NO: 3 - DNA, 1232 bp, Porcine
  allele (1078)..(1078): c/g
gcctcgccctctccgacctgatccagaaatacttcgtgtcccccacgctgtttcgtgtgatccgcctggccaggatcggtcgcgtcctgcggctgatccgcggggccaagggcatccggacgctgctctttgccctcatgatgtcgctgcccgccctcttcaacatcggcctgctcctcttcctggtcatgttcatctactccatcttcggcatgtccaacttcgcctacgtcaagaaggagtcgggcatcgacgacatgttcaacttcgagaccttcggcaacagcatcatctgcctcttcgagatcacgacgtcggcgggctgggacgggctgctcaaccccatcctcaacagcgggccccccgactgcgaccccacgctggagaacccgggcaccagcgtccggggcgactgcggcaacccgtccatcggcatctgcttcttctgcagctacatcatcatctccttcctcatcgtggtcaacatgtacatcgccatcatcctggagaacttcaacgtggccacggaggagagcagcgagcccctcggggaggacgacttcgagatgttctacgagacgtgggagaagttcgaccccgacgccacgcagttcatcgactacagccgcctctcggacttcgtggacaccctgcaggagccgctgaggatcgccaagcccaacaagatcaagctcatcaccatggacctgcccatggtgccgggggacaagatccactgcctggacatcctcttcgccctgaccaaggaggtcctgggcgactctggggagatggacgccctcaaggagaccatggaggagaagttcatggctgccaacccctccaaggtctcctacgagcccatcaccaccacgctcaagaggaagcacgaggaggtgtgcgccatcaagatccagagggcctaccgccggcacctgctccagcgctccgtgaagcaggcgtcctacatgtaccgccagagccacgacggcggtggcggcggggacggggcccccgagaaggaggggctgattgccgacaccatgagcaagatgtacggccaggagaacgggaacaccagtgcgcagagccagggggaggcgaggggctggacaggggcccccgaacctgccatggggctcgtgctcatcagcccctcagaggccgccctcccgcccaccccacccctggggcagactgtgcgccccggggtcaaagagtcacttgtctag

SEQ ID NO: 4 - DNA, 239 bp, Porcine
  allele (78)..(78): g/a
agctggaagaggcccaccagaagtgcccaccgtggtggtacaagtgctcccacaaagtgctcatatggaactgctgcggcccctggatgaagttcaagaacatcatccacctgattgtcatggaccccttcgtggacctgggcatcaccatctgcatcgtgctcaacaccctcttcatggccatggagcattaccccatgaccgaggagtttgacgccgtcctcaccgtgggcaacttg

SEQ ID NO: 5 - DNA, 621 bp, Porcine
  allele (406)..(406): g/a
ggccccgagagcctgcgccccttcacccgggagtccctggctgccatagagcagcgggtggtggaggaggaggcccggcagcagcggaacaagcagatggagatcgaggagccagaacggaagcctcgcagcgacctggaggctggcaaaaacctgccccttatctatggggaccccccacccgaggtcatcggcatccctctggaggacctggatccctactacagcgacaagaaggtcagggcctgggcgggttcctctgtctgtctgtccgtcgtcatctgtctgcctgtcccgggcctcacagctctctccctgcttcagaccttcatcgtgctcaacaagggcaaggccatcttccgcttctctgccacgcctgctctctacgtgctgagccccttcagcgtcgtcaggcgcagcgccatcaaggtgctcatccactcatatcctgccagagtcgggcgagcgccgggctgggaaaaggcaggggaggggtttggggacaggccaaacggggtgctctggccggggagcacctccctccccacctgctctctccctttccttgaccccccccccaacgctgttcagcatgttcatcatgatcacgatcctgaccaa

SEQ ID NO: 6 - DNA, 525 bp, Porcine
  allele (96)..(96): R = g or a
gtgcctgtgtggagcggagtaaatgttgctggtgtctccctgaagaatctgcaccctgaattaggcactgatgcagataaggaacactggaaagcrgttcacaaacaggtggtggacaggtaatagatctcataatttgtaatgtgaaaggttaaaatttattattttatttaaaaaactaaaagtttaataatatttgcattcgatttactctgtcagaaaacttgttttctaaagctttttaaaatatcatactataaaaaggtaaaggcattaaaaattacagacatttataaatgctacagtctatctttatttgctgtaattctctatagtatgataaatctttgtgtttgtaatgtaaactaataagataaaagaggagttcctgtcgtggctcagtggaaactattctgactagtatccatgaggatgtaagtttgatccctgaccttgctcagtggattaaggatcaggcattgctgtgagctgtggtgtaggttacaacgcggctcggatctgggg

SEQ ID NO: 7 - DNA, 20 bp, Porcine
cagcccatacaaggccatgg

SEQ ID NO: 8 - DNA, 37 bp, Porcine
ctggctgggctgtgctggaatatcctggaggcgacac

SEQ ID NO: 9 - DNA, 20 bp, Porcine
tctgacccagaggtgtcaag

SEQ ID NO: 10 - DNA, 20 bp, Porcine
cagcccacggtcatgatgaa

SEQ ID NO: 11 - DNA, 41 bp, Porcine
cgagggctgttaaaggccaaggctcctttctccagggacac

SEQ ID NO: 12 - DNA, 41 bp, Porcine
atcatgcgcttcaccgactgggagaaagagcctctccgtcc

SEQ ID NO: 13 - DNA, 23 bp, Porcine
acgaggaggtgtgcgccatcaag

SEQ ID NO: 14 - DNA, 22 bp, Porcine
atgagcacgagccccatggcag

SEQ ID NO: 15 - DNA, 19 bp, Porcine
ggaagaggcccaccagaag

SEQ ID NO: 16 - DNA, 19 bp, Porcine
caagttgcccacggtgagg

SEQ ID NO: 17 - DNA, 20 bp, Porcine
cgtcgtcatctgtctgcctg

SEQ ID NO: 18 - DNA, 18 bp, Porcine
atggcgctgcgcctgtcg

SEQ ID NO: 19 - DNA, 20 bp, Porcine
gtgtggagcggagtaaatgt

SEQ ID NO: 20 - DNA, 21 bp, Porcine
ccccagatccgagccgcgttg